(19) United States
(12) Patent Application Publication
(10) Pub. No.: US 2009/0118365 A1
(43) Pub. Date: May 7, 2009

(54) Use of Prodrugs of GABA B Agonists for Treating Neuropathic and Musculoskeletal Pain

(75) Inventors: Joseph C. Benson, III, San Jose, CA (US); Peter A. Virsik, Portola Valley, CA (US); Daniel M. Canafax, Half Moon Bay, CA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: XenoPort, Inc, Santa Clara, CA (US)

(21) Appl. No.: 12/266,169

(22) Filed: Nov. 6, 2008

Related U.S. Application Data
(60) Provisional application No. 60/985,909, filed on Nov. 6, 2007.

(51) Int. Cl. A61K 31/27; A61K 31/195; A61P 25/00

(52) U.S. Cl. 514/487; 514/567

(57) ABSTRACT

Methods of treating neuropathic pain, musculoskeletal pain, and back spasm associated with musculoskeletal pain in a patient comprising orally administering a therapeutically effective dose of a prodrug of a GABA_{B} agonist having a high oral bioavailability of the corresponding GABA_{B} agonist are disclosed.

This application claims the benefit under 35 U.S.C. § 119(e) of U.S. Provisional Application Ser. No. 60/985,909, filed Nov. 6, 2007, which is incorporated by reference herein in its entirety.

### FIELD

Disclosed herein are methods of treating neuropathic pain, musculoskeletal pain, and back spasms associated with musculoskeletal pain using prodrugs of GABA_{B} agonists having a high oral bioavailability of the corresponding GABA_{B} agonist.

### BACKGROUND

(±)-4-Amino-3-(4-chlorophenyl)butanoic acid (baclofen) (1) is an analog of gamma-aminobutyric acid (i.e., GABA) that selectively activates GABA_{B} receptors, resulting in neuronal hyperpolarization. GABA_{B} receptors are located in laminae I-IV of the spinal cord, where primary sensory fibers terminate. These G-protein coupled receptors activate conductance by K⁺-selective ion channels and can reduce currents mediated by Ca²⁺ channels in certain neurons. Baclofen has a presynaptic inhibitory effect on the release of excitatory neurotransmitters and also acts postsynaptically to decrease motor neuron firing.

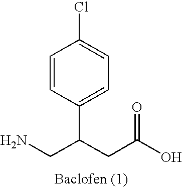

Baclofen is a GABA_{B} receptor agonist that has been used in the United States since 1977 for alleviating the signs and symptoms of spasticity resulting from multiple sclerosis or spinal cord injury. The mechanism of action of baclofen in spasticity appears to involve agonism at GABA_{B} receptors of the spinal cord. Baclofen is also useful in controlling gastro-esophageal reflux disease (van Herwaarden et al., Aliment. Pharmacol. Ther. 2002, 16, 1655-1662; Ciccaglione et al., Gut 2003, 52, 464-470; Andrews et al., U.S. Pat. No. 6,117,908; and Fara et al., WO 02/096404); in promoting alcohol abstinence in alcoholics (Gessa et al., WO 01/26638); in promoting smoking cessation (Gessa et al., WO 01/08675); in reducing addiction liability of narcotic agents (Robson et al., U.S. Pat. No. 4,126,684); in the treatment of emesis (Bountra et al., U.S. Pat. No. 5,719,185); and as an anti-tussive for the treatment of cough (Kreutner et al., U.S. Pat. No. 5,006,560).

Baclofen may be administered orally or by intrathecal delivery through a surgically implanted programmable pump. The drug is rapidly absorbed from the gastrointestinal tract and has an elimination half-life of approximately 3-4 hours. Baclofen is partially metabolized in the liver but is largely excreted by the kidneys unchanged. The short half-life of baclofen necessitates frequent administration with typical oral dosing regimens ranging from about 10 mg to about 80 mg of three or four divided doses daily. Plasma baclofen concentrations of about 80 ng/mL to about 400 ng/mL result from these therapeutically effective doses in patients for the treatment of spasticity. When baclofen is given orally, sedation is a common side effect, particularly at elevated doses. Impairment of cognitive function, confusion, memory loss, dizziness, weakness, ataxia, and orthostatic hypotension are other commonly encountered baclofen side-effects.

Intrathecal administration is often recommended for patients who find the adverse effects of oral baclofen intolerable. The intrathecal use of baclofen permits effective treatment of spasticity with doses less than 1/100^{th} of those required orally because administration directly into the spinal subarachnoid space permits immediate access to GABA_{B} receptor sites in the dorsal horn of the spinal cord. Surgical implantation of a pump is, however, inconvenient and a variety of mechanical and medical complications can arise (e.g., catheter displacement, kinking or blockage, pump failure, sepsis, and deep vein thrombosis) including the potential for overdose and abrupt cessation of drug delivery. Acute discontinuation of baclofen therapy (e.g., in cases of mechanical failure) may cause serious withdrawal symptoms such as hallucinations, confusion, agitation, and seizures.

While the clinically prescribed baclofen product (Lioresal™) is available only as a racemate, the GABA_{B} receptor agonist activity resides entirely in one enantiomer, R-(−)-baclofen (2) (also termed L-baclofen).

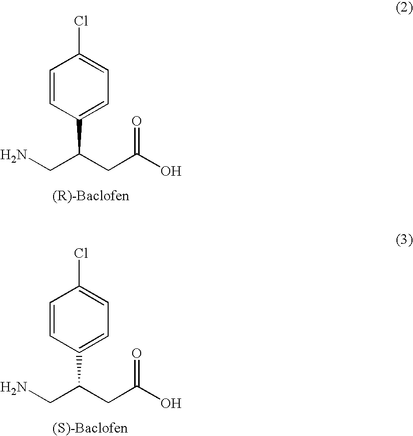

The other isomer, (S)-baclofen (3), antagonizes the action of (R)-baclofen at GABA_{B} receptors and the antinociceptive activity of (R)-baclofen in the rat spinal cord. Orally administered (R)-baclofen is reported to be about 5-fold more potent than orally administered racemic baclofen, with an (R)-baclofen regimen of 2 mg t.i.d being equivalent to racemic baclofen at 10 mg t.i.d. Moreover, the side effect profile following administration of (R)-baclofen, has been shown to be significantly reduced relative to an equally efficacious dose of racemic baclofen.

Baclofen, a zwitterionic amino acid, lacks the requisite physicochemical characteristics for effective passive permeability across cellular membranes. Passage of the drug across the gastrointestinal tract and the blood-brain barrier (BBB) are mediated primarily by active transport processes rather than by passive diffusion. Baclofen is a substrate for active transport mechanisms shared by neutral α-amino acids such as leucine, and β-amino acids such as β-alanine and taurine. Transport across the BBB is stereoselective, with preferential uptake of the active R-enantiomer (2). In addition, organic anion transporters localized in capillary endothelial cells of the blood-brain barrier have been implicated in efflux of baclofen from the brain.

Sustained released oral dosage formulations are a conventional solution to the problem of rapid systemic drug clearance, as is well known in the art. Successful application of these technologies depends on the drug of interest having an effective level of absorption from the large intestine (also referred to herein as the colon), where the dosage form spends a majority of time during passage through the gastrointestinal tract. Baclofen is poorly absorbed following administration into the colon in animal models, presumably because the transporter proteins mediating baclofen absorption in the upper region of the small intestine are not expressed in the large intestine. Development of an oral, controlled release formulation of baclofen should considerably improve the convenience, efficacy, and side effect profile of baclofen therapy. However, the rapid passage of conventional dosage forms through the proximal absorptive region of the small intestine has thus far prevented the successful application of sustained release technologies to this drug. A number of exploratory delivery technologies that rely on either mucoadhesion or gastric retention have been suggested to achieve sustained delivery of baclofen; however to date none of these appear to be able to achieve sustained blood levels of baclofen in human subjects.

Recently, Gallop et al. have developed new prodrugs of (R)-baclofen and baclofen analogs that are well absorbed in the large intestine/colon and hence suitable for oral sustained release formulations, thus improving the convenience, efficacy and side effect profile of baclofen therapy (Gallop et al., U.S. Pat. No. 7,109,239, U.S. Pat. No. 7,227,028, U.S. Pat. No. 7,300,131, and US 2008/0096960; Leung et al., US 2008/0206332; Cundy, U.S. application Ser. No. 12/139,057 filed Jun. 13, 2008, and Sastry et al., U.S. application Ser. No. 12/024,830 filed Feb. 1, 2008; each of which is incorporated by reference herein in its entirety). For example, (3R)-4-{[(1S)-2-methyl-1-(2-methylpropanoyloxy)propoxy]carbonylamino}-3-(4-chlorophenyl)butanoic acid, (4),

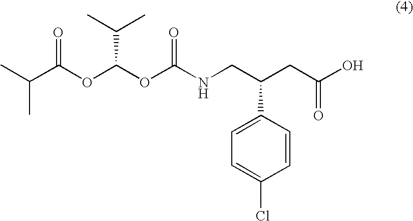

a prodrug of the GABA analog, (R)-baclofen (2), exhibits high bioavailability as (R)-baclofen when dosed either orally or directly into the colon of a mammal (Gallop et al., U.S. Pat. No. 7,109,239). These prodrugs of GABA_{B} agonists provide improved oral bioavailability of the corresponding GABA_{B} agonist and can also facilitate oral GABA_{B} agonist regimens capable of providing therapeutically effective blood concentrations of GABA_{B} agonists appropriate for treating chronic diseases and disorders. Furthermore, certain prodrugs of GABA_{B} agonists exhibit high absorption from the colon and therefore can facilitate administration of GABA_{B} agonists in sustained release dosage forms.

### SUMMARY

Prodrugs of GABA_{B} agonists that provide a high oral bioavailability of GABA_{B} agonists and are colonically absorbable, such as the prodrugs GABA_{B} agonists disclosed by Gallop et al. enable the use of orally administered GABA_{B} agonists for treating neuropathic and musculoskeletal pain, and in particular back spasms associated with musculoskeletal pain potentially without the inconvenience and/or adverse effect profile associated with currently used pharmaceuticals for treating neuropathic and musculoskeletal pain. Accordingly, methods of treating neuropathic pain, musculoskeletal pain, and back spasms associated with musculoskeletal pain in a patient are disclosed comprising orally administering to a patient in need of such treatment a therapeutically effective dose of a colonically absorbable prodrug of a GABA_{B} agonist that is capable of providing a high oral bioavailability of the corresponding GABA_{B} agonist. These and other features of the present disclosure are set forth herein.

### DETAILED DESCRIPTION

### Definitions

A dash (“-”) that is not between two letters or symbols is used to indicate a point of attachment for a substituent. For example, —CONH₂ is attached through the carbon atom.

“Adverse drug effects” refers to drug effects that are unwanted, unpleasant, noxious, and/or potentially harmful. Adverse drug effects can be mild such as digestive disturbance, headaches, fatigue, vague muscle aches, malaise, and changes in sleep patterns. Moderate adverse drug effects represent reactions that a person considers annoying, distressing, or intolerable such as skin rashes, visual disturbances, muscle tremor, difficulty with urination, perceptible changes in mood or mental function, and certain changes in blood components. Examples of severe adverse drug effects include reactions that may be life threatening, that result in persistent or significant disability or hospitalization, and/or that cause a birth defect. Examples of adverse effects known to be associated with baclofen therapy include sedation, impairment of cognitive function, confusion, memory loss, dizziness, weakness, ataxia, blurred or double vision, nausea, shortness of breath, convulsions, and orthostatic hypotension.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group comprises from 1 to 20 carbon atoms (C₁₋₂₀), in certain embodiments, from 1 to 10 carbon atoms (C₁₋₁₀), from 1 to 8 carbon atoms (C₁₋₈), from 1 to 6 carbon atoms (C₁₋₆), from 1 to 4 carbon atoms (C₁₋₄), and in certain embodiments, from 1 to 3 carbon atoms (C₁₋₃).

“Acyl” by itself or as part of another substituent refers to a radical —C(O)R⁷⁰, where R⁷⁰ is hydrogen, alkyl, heteroalkyl, cycloalkyl, heterocycloalkyl, cycloalkylalkyl, heterocycloalkylalkyl, aryl, heteroaryl, arylalkyl, or heteroarylalkyl, which can be substituted, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR³¹ where R³¹ is chosen from alkyl, heteroalkyl, cycloalkyl, heterocycloalkyl, cycloalkylalkyl, heterocycloalkylalkyl, aryl, heteroaryl, arylalkyl, and heteroarylalkyl, as defined herein. Examples of alkoxy groups include, but are not limited to, methoxy, ethoxy, propoxy, butoxy, cyclohexyloxy, and the like. In certain embodiments, an alkoxy group is C₁₋₁₈ alkoxy, in certain embodiments, C₁₋₁₂ alkoxy, in certain embodiments, C₁₋₈ alkoxy, in certain embodiments, C₁₋₆ alkoxy, in certain embodiments, C₁₋₄ alkoxy, and in certain embodiments, C₁₋₃ alkoxy.

“Alkoxycarbonyl” by itself or as part of another substituent refers to a radical —C(O)OR⁷² where R⁷² represents an alkyl, as defined herein. Examples of alkoxycarbonyl groups include, but are not limited to, methoxycarbonyl, ethoxycarbonyl, propoxycarbonyl, and butoxycarbonyl, and the like.

“Amino” refers to the radical —NH₂.

“Anesthesia” as used herein includes general anesthesia and deep sedation. General anesthesia is a drug-induced loss of consciousness during which patients are not arousable, even by painful stimulation. Deep sedation is a drug-induced depression of consciousness during which patients cannot be easily aroused but respond purposefully following repeated or painful stimulation. Reflex withdrawal from a painful stimulus is not a purposeful response. In deep sedation the ability of a patient to maintain ventilatory function may be impaired, while in general anesthesia, the ability to independently maintain ventilatory function is often impaired and often requires intervention in maintaining an open airway.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), and in certain embodiments, from 6 to 10 carbon atoms (C₆₋₁₀). Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound in a biological fluid in a patient as a function of time following administration of the compound to the patient. Examples of biological fluids include plasma and blood. The AUC can be determined by measuring the concentration of a compound in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the disclosure herein, an AUC for a GABA_{B} agonist can be determined by measuring the concentration of the GABA_{B} agonist in the plasma or blood of a patient following oral administration of a dosage form comprising a corresponding prodrug of the GABA_{B} agonist.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to peak concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the highest drug concentration observed in the plasma or blood following a dose of drug.

Compounds encompassed by structural Formulae (I)-(VI) disclosed herein include any specific compounds within these formulae. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to those skilled in the art.

Compounds of Formulae (I)-(VI) include optical isomers of compounds of Formulae (I)-(VI), racemates thereof, and other mixtures thereof. In such embodiments, the single enantiomers or diastereomers, i.e., optically active forms, can be obtained by asymmetric synthesis or by resolution of the racemates. Resolution of enantiomers and diastereomers may be accomplished, for example, by conventional methods such as crystallization in the presence of a resolving agent, or chromatography, using, for example a chiral high-pressure liquid chromatography (HPLC) column. In addition, compounds of Formulae (I)-(VI) include Z- and E-forms (e.g., cis- and trans-forms) of compounds with double bonds.

In embodiments in which compounds of Formulae (I)-(VI) exist in various tautomeric forms, compounds of the present disclosure include all tautomeric forms of the compound. The compounds of Formulae (I)-(VI) may also exist in several tautomeric forms including the enol form, the keto form, and mixtures thereof. Accordingly, the chemical structures depicted herein encompass all possible tautomeric forms of the illustrated compounds. The compounds of Formulae (I)-(VI) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated, or N-oxides. Thus, when reference is made to compounds of the present disclosure, such as compounds of Formula (I)-(VI), it is understood that a compound also implicitly refers to free acids, salts, solvates, hydrates, and combinations of any of the foregoing. Certain compounds may exist in multiple crystalline, cocrystalline, or amorphous forms. Compounds of Formula (I)-(VI) include or pharmaceutically acceptable solvates of a free acid or salt form of any of the foregoing, hydrates of a free acid or salt form of any of the foregoing, as well as crystalline forms of any of the foregoing.

In general, all physical forms are equivalent for the uses contemplated herein and are intended to be within the scope of the present disclosure.

Compounds of Formula (I)-(VI) may be solvates. The term “solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

Further, when partial structures of the compounds are illustrated, an asterisk (*) indicates the point of attachment of the partial structure to the rest of the molecule.

“Colonically absorbable prodrug of a GABA_{B} agonist” means a prodrug of a GABA_{B} agonist, as defined herein, which provides an AUC of the corresponding GABA_{B} agonist following colonic administration of the prodrug that is at least two times greater than the AUC of the GABA_{B} agonist following colonic administration of an equivalent amount of the GABA_{B} agonist itself.

“Controlled delivery” means continuous or discontinuous release of a compound over a prolonged period of time, wherein the compound is released at a controlled rate over a controlled period of time in a manner that provides for upper gastrointestinal and lower gastrointestinal tract delivery, coupled with improved compound absorption as compared to the absorption of the compound in an immediate release oral dosage form.

“Corresponding GABA_{B} agonist” means a compound of Formula (IV) having the same R⁵ as the prodrug of a GABA_{B} agonist of Formula (I), Formula (II), or Formula (III). Similarly, a “corresponding prodrug of a GABA_{B} agonist” means a compound of Formula (I), Formula (II), or Formula (III) having the same R⁵ group as the GABA_{B} agonist of Formula (IV).

“Cycloalkoxycarbonyl” by itself or as part of another substituent refers to a radical —C(O)OR⁷⁶ where R⁷⁶ represents an cycloalkyl group as defined herein. Examples of cycloalkoxycarbonyl groups include, but are not limited to, cyclobutyloxycarbonyl, cyclohexyloxycarbonyl, and the like.

“Cycloalkyl” by itself or as part of another substituent refers to a partially saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₃₋₁₂ cycloalkyl or C₅₋₁₂ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“Disease” refers to a disease, disorder, condition, or symptom.

“Dosage form” means a pharmaceutical composition in a medium, carrier, vehicle, or device suitable for administration to a patient.

“GABA analog” means a compound having the following structure:

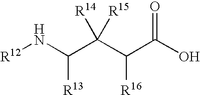

wherein: R¹² is hydrogen, or R¹² and R¹⁶ together with the atoms to which they are bonded form a ring chosen from an azetidine, substituted azetidine, pyrrolidine, and substituted pyrrolidine ring;

R¹³ and R¹⁶ are independently chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl; and

R¹⁴ and R¹⁵ are independently chosen from hydrogen, alkyl, substituted alkyl, acyl, substituted acyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R¹⁴ and R¹⁵ together with the carbon atom to which they are bonded form a ring chosen from a cycloalkyl, substituted cycloalkyl, cycloheteroalkyl, substituted cycloheteroalkyl, and bridged cycloalkyl ring.

In certain embodiments of a GABA analog, each substituent is independently chosen from halogen, —NH₂, —OH, —CN, —COOH, —C(O)NH₂, —C(O)OR¹⁰, and —NR¹⁰₃⁺ wherein each R¹⁰ is independently C₁₋₃ alkyl.

In certain embodiments of a GABA analog, R¹² is hydrogen.

In certain embodiments of a GABA analog, R¹² is hydrogen, R¹³ is hydrogen, R¹⁶ is hydrogen, and R¹⁴ and R¹⁵ together with the carbon atom to which they are bonded form a cyclohexyl ring.

In certain embodiments of a GABA analog, R¹² is hydrogen, R¹³ is hydrogen, R¹⁶ is hydrogen, R¹⁴ is hydrogen, and R¹⁵ is isobutyl.

In certain embodiments, a GABA analog is chosen from gabapentin and pregabalin. Furthermore, a number of GABA analogs with considerable pharmaceutical activity have been synthesized in the art (Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, WO 92/09560; Silverman et al., WO 93/23383; Horwell et al., WO 97/29101, Horwell et al., WO 97/33858; Horwell et al., WO 97/33859; Bryans et al., WO 98/17627; Guglietta et al., WO 99/08671; Bryans et al., WO 99/21824; Bryans et al., WO 99/31057; Belliotti et al., WO 99/31074; Bryans et al., WO 99/31075; Bryans et al., WO 99/61424; Bryans et al., WO 00/15611; Bryans, WO 00/31020; Bryans et al., WO 00/50027; and Bryans et al., WO 02/00209); WO 98/23383; Bryans et al., J. Med. Chem. 1998, 41, 1838-1845; Bryans et al., Med. Res. Rev. 1999, 19, 149-177, US Guglietta et al., WO 99/08670; Bryans et al., WO 99/21824; US and Bryans et al., UK GB 2 374 595). Pharmaceutically important GABA analogs include, for example, gabapentin, pregabalin, vigabatrin, and baclofen.

“GABA_{B} receptor” includes the subtypes of presynaptic receptors comprising heteroreceptors as well as autoreceptors, and postsynaptic receptors that are inhibited by GABA and are coupled through G-proteins to Ca²⁺ or K⁺ channels. The GABA_{B} receptor exists as a heterodimer with two subunits, GABA_{BR1} and GABA_{BR2}, which provide different functions but are mutually dependent. The GABA_{BR1} subunit contains the GABA-binding domain and the GABA_{BR2} subunit provides the G-protein-coupling mechanism and also incorporates an allosteric modulatory site within its heptahelical structure. Four different functional isoforms of the human GABA_{BR1} subunit have been identified; however there is no unequivocal evidence for distinct GABA_{BR2} receptor subtypes. The variants of the GABA_{BR1} subunit do not appear to have significant pharmacological differences with respect to activator or inhibitor binding.

The terms “GABA_{B} receptor agonist” and “GABA_{B} agonist” are used interchangeably herein and both mean compounds, such as (R)-baclofen, that elicit a positive effect in any of the GABA_{B} agonist functional assays described herein such as the cAMP, Ca²⁺, and electrophysiology in vitro assay, the hypothermia animal model, or in any other accepted functional assay for determining GABA_{B} receptor agonist activity known in the art. In certain embodiments, a GABA_{B} agonist means a compound of Formula (IV):

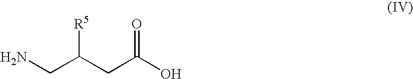

where R⁵ is chosen from substituted aryl, heteroaryl and substituted heteroaryl. In certain embodiments of a compound of Formula (IV), R⁵ is chosen from 4-chlorophenyl, (3R)-4-chlorophenyl, 2-chlorophenyl, 4-fluorophenyl, thien-2-yl; 5-chlorothien-2-yl, 5-bromothien-2-yl, 5-methylthien-2-yl, and 2-imidazolyl. In certain embodiments of a compound of Formula (IV), R⁵ is chosen from 4-chlorophenyl, (3R)-4-chlorophenyl, 2-chlorophenyl, 4-fluorophenyl. In certain embodiments, R⁵ is 4-chlorophenyl and the compound of Formula (IV) is (R)-baclofen, (R)-4-amino-3-(4-chlorophenyl)butanoic acid.

“Halogen” refers to a fluoro, chloro, bromo, or iodo group.

“Heteroalkyl” by itself or as part of another substituent refer to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, —NR³⁷, ═N—N═, —N═N—, —N═N—NR³⁷, —PR³⁷—, —P(O)₂—, —POR³⁷—, —O—P(O)₂—, —SO—, —SO₂—, —Sn(R³⁷)₂—, and the like, where each R³⁷ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₅₋₁₂ heteroaryl, substituted C₅₋₁₂ heteroaryl, C₆₋₁₈ heteroarylalkyl, or substituted C₆₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R³⁷ is independently chosen from hydrogen and C₁₋₃ alkyl. In certain embodiments, a heteroatomic group is chosen from —O—, —S—, —NH—, —N(CH₃)—, and —SO₂—.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which can be aromatic or non-aromatic in which at least one ring atom is a heteroatom. Heteroaryl encompasses 5- to 12-membered aromatic, such as 5- to 7-membered, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. In certain embodiments, a heteroaryl group is C₅₋₁₂ heteroaryl, C₅₋₁₀ heteroaryl, and in certain embodiments, C₅₋₆ heteroaryl. The ring of a C₅₋₁₀ heteroaryl has from 4 to 9 carbon atoms, with the remainder of the atoms in the ring being heteroatoms.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl. In certain embodiments, a heteroarylalkyl group is C₆₋₁₈ heteroarylalkyl and in certain embodiments, C₆₋₁₀ heteroarylalkyl.

“Heterocycloalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. Heterocycloalkyl includes nonaromatic heterocycloalkyl fused ring systems. In certain embodiments, a heterocycloalkyl group is a C₃₋₁₂ heterocycloalkyl, C₃₋₁₀ heterocycloalkyl, and in certain embodiments C₃₋₈ heterocycloalkyl.

“Heterocycloalkylalkyl” by itself or as part of another substituent refers to an acyclic alkyl radical in which one of the hydrogen atoms bonded to a carbon atom, typically a terminal or sp³ carbon atom, is replaced with a heterocycloalkyl group. Where specific alkyl moieties are intended, the nomenclature heterocycloalkylalkanyl, heterocycloalkylalkenyl, or heterocycloalkylalkynyl is used. In certain embodiments, a heterocycloalkylalkyl group is a 6- to 30-membered heterocycloalkylalkyl, e.g., the alkanyl, alkenyl, or alkynyl moiety of the heterocycloalkylalkyl is 1- to 10-membered and the heterocycloalkyl moiety is a 5- to 20-membered heterocycloalkyl, and in certain embodiments, 6- to 20-membered heterocycloalkylalkyl, e.g., the alkanyl, alkenyl, or alkynyl moiety of the heterocycloalkylalkyl is 1- to 8-membered and the heterocycloalkyl moiety is a 5- to 12-membered heterocycloalkyl. In certain embodiments, a heterocycloalkylalkyl group is a C₄₋₁₈ heterocycloalkylalkyl, C₄₋₁₂ heterocycloalkylalkyl, and in certain embodiments C₄₋₁₀ heterocycloalkylalkyl.

“Hydroxyl” refers to the group —OH.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” includes animals and mammals, such as for example, humans.

“Pharmaceutical composition” refers to at least one compound of Formula (I), Formula (II), or Formula (III) and at least one pharmaceutically acceptable vehicle, with which the at least one compound of Formula (I), Formula (II), or Formula (III) is administered to a patient.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include: (I) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt, and in certain embodiments, the sodium salt. Pharmaceutically acceptable salts may be prepared by the skilled chemist, by treating, for example, a compound of Formulae (I)-(VI) with an appropriate base in a suitable solvent, followed by crystallization and filtration.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound of Formulae (I)-(III) may be administered to a patient and which does not destroy the pharmacological activity thereof, and which is nontoxic when administered in doses sufficient to provide a therapeutically effective amount of the compound.

“Prodrug” refers to a derivative of a drug molecule that requires a transformation within the body to release the active drug. Prodrugs are frequently, although not necessarily, pharmacologically inactive until converted to the parent drug. Prodrugs may be obtained by bonding a promoiety (defined herein) typically via a functional group, to a drug. For example, referring to compounds of Formula (I), Formula (II), and Formula (III) the promoiety is bonded to the GABA_{B} agonist via an amide bond. Compounds of Formula (I), Formula (II), and Formula (III) are prodrugs of GABA_{B} agonists that can be metabolized within a patient's body to release the corresponding GABA_{B} agonist.

“Prodrug of a GABA_{B} agonist” or “prodrug of a GABA_{B} agonist provided by the present disclosure” refers to a compound in which a promoiety that is cleavable in vivo, and is covalently bound to a GABA_{B} agonist. In certain embodiments, a prodrug may be actively transported by transporters expressed in the enterocytes lining the gastrointestinal tract such as, for example, the PEPT1 transporter. Prodrugs of GABA_{B} agonists can be stable in the gastrointestinal tract and following absorption are cleaved in the systemic circulation to release the corresponding GABA_{B} agonist. In certain embodiments, a prodrug of a GABA_{B} agonist provides a greater oral bioavailability of the corresponding GABA_{B} agonist compared to the oral bioavailability of the GABA_{B} agonist when administered as a uniform liquid immediate release formulation. In certain embodiments, a prodrug of a of GABA_{B} agonist provides a high oral bioavailability of the corresponding GABA_{B} agonist, for example, exhibiting a GABA_{B} agonist oral bioavailability that is at least 2 times greater than the oral bioavailability of the same GABA_{B} agonist when orally administered in an equivalent dosage form, and in certain embodiments at least 10 times greater than the oral bioavailability of the same GABA_{B} agonist when orally administered in an equivalent dosage form. In certain embodiments, a prodrug of a GABA_{B} agonist is a compound having a structure encompassed by any one of Formulae (I)-(III) and/or compound (4), or a pharmaceutically acceptable salt of any of the foregoing. In certain embodiments, a prodrug of a GABA_{B} agonist is compound (4) or a pharmaceutically acceptable salt thereof.

“Promoiety” refers to a chemical group, i.e. moiety, bonded to a drug, typically to a functional group of the drug, via bond(s) that are cleavable under specified conditions of use. The bond(s) between the drug and promoiety may be cleaved by enzymatic or non-enzymatic means. Under conditions of use, for example following administration to a patient, the bond(s) between the drug and promoiety may be cleaved to release the parent drug. Cleavage of the promoiety may proceed spontaneously, such as via a hydrolysis reaction, or may be catalyzed or induced by another agent, such as by an enzyme, by light, by acid, or by a change of or exposure to a physical or environmental parameter such as a change of temperature, pH, etc. The agent may be endogenous to the conditions of use, such as an enzyme present in the systemic circulation of a patient to which the prodrug is administered or the acidic conditions of the stomach or the agent may be supplied exogenously. As an example, for a prodrug of Formula (III), the promoiety is:

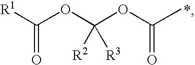

where R¹, R², and R³ are as defined herein, and the drug is (R)-baclofen.

“Sedation” as used herein refers to minimal sedation and/or moderate sedation (American Society of Anesthesiologists, Anesthesiology 2002, 96, 1004-17). Minimal sedation, also referred to as anxiolysis, is a minimally depressed level of consciousness that retains the patient's ability to independently and continuously maintain an airway and respond appropriately to physical stimulation or verbal command that is produced by a pharmacological or non-pharmacological method or combination thereof. Although cognitive function and coordination may be modestly impaired, ventilatory and cardiovascular functions are unaffected. When the intent is minimal sedation in adults, the appropriate dosing is no more than the maximum recommended dose that can be prescribed for unmonitored home use, e.g., a maximum recommended therapeutic dose. Moderate sedation is a drug-induced depression of consciousness during which patients respond purposefully to verbal commands, either alone or accompanied by light tactile stimulation. No intervention is required to maintain a patient's airway. Sedation is a continuum and it is not always possible to predict how an individual patient will respond. A sedative dose can be determined by incremental dosing, administering multiple doses of a drug, such as a prodrug of a GABA_{B} agonist provided by the present disclosure, until a desired effect is reached. A variety of scales can be used to assess sedation including, for example, the Ramsay Scale and the Observer's Assessment of Alertness/Sedation Scale. Objective measures of sedation include measurement of electroencephalogram parameters such as the Bispectral Index version XP and the Patient State Analyzer. In certain embodiments, sedation refers to minimal sedation, and in certain embodiments, moderate sedation.

“Solvate” refers to a molecular complex of a compound with one or more solvent molecules in a stoichiometric or non-stoichiometric amount. Such solvent molecules are those commonly used in the pharmaceutical art, which are known to be innocuous to recipient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a complex where the one or more solvent molecules are water including monohydrates and hemi-hydrates.

“Substantially one diastereomer” refers to a compound containing two or more stereogenic centers such that the diastereomeric excess (d.e.) of the compound is greater than or about at least 90%. In certain embodiments, the d.e. is, for example, greater than or at least about 91%, about 92%, about 93%, about 94%, about 95%, about 96%, about 97%, about 98%, or about 99%.

“Substituted” refers to a group in which one or more hydrogen atoms are independently replaced with the same or different substituent(s). Examples of substituents include, but are not limited to, -Q, —R⁶⁰, —O⁻, —OH, ═O, —OR⁶⁰, —SR⁶⁰, —S—, ═S, —NR⁶⁰R⁶¹, ═NR⁶⁰, —CX₃, —CN, —CF₃, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, C(O)NR⁶⁰R⁶¹, C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, —C(NR⁶²)NR⁶⁰R⁶¹, —S(O)₂, NR⁶⁰R⁶¹, —NR⁶³S(O)₂R⁶⁰, —NR⁶³C(O)R⁶⁰, and —S(O)R⁶⁰ where each Q is independently a halogen; each R⁶⁰ and R⁶¹ are independently chosen from hydrogen, alkyl, substituted alkyl, alkoxy, substituted alkoxy, cycloalkyl, substituted cycloalkyl, heterocycloalkyl, substituted heterocycloalkyl, aryl, substituted aryl, heteroaryl, substituted heteroaryl, arylalkyl, substituted arylalkyl, heteroarylalkyl, and substituted heteroarylalkyl; or R⁶⁰ and R⁶¹ together with the nitrogen atom to which they are bonded form a ring chosen from a heterocycloalkyl, substituted heterocycloalkyl, heteroaryl, and substituted heteroaryl ring, and R⁶² and R⁶³ are independently chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroaryl, substituted heteroaryl, heteroarylalkyl, and substituted heteroarylalkyl, or R⁶² and R⁶³ together with the atom to which they are bonded form a ring chosen from a heterocycloalkyl, substituted heterocycloalkyl, heteroaryl, and substituted heteroaryl ring. In certain embodiments, a tertiary amine or aromatic nitrogen may be substituted with one or more oxygen atoms to form the corresponding nitrogen oxide.

In certain embodiments, substituted aryl and substituted heteroaryl include one or more of the following substitute groups: F, Cl, Br, C₁₋₃ alkyl, substituted C₁₋₃ alkyl, C₁₋₃ alkoxy, substituted C₁₋₃ alkoxy, —S(O)₂NR⁶⁰R⁶¹, —NR⁶⁰R⁶¹, —CF₃, —OCF₃, —CN, —NR⁶⁰S(O)₂R⁶¹, —NR⁶⁰C(O)R⁶¹, C₅₋₁₀ aryl, substituted C₅₋₁₀ aryl, C₅₋₁₀ heteroaryl, substituted C₅₋₁₀ heteroaryl, —C(O)OR⁶⁰, —NO₂, —C(O)R⁶⁰, —C(O)NR⁶⁰R⁶¹, —OCHF₂, C₁₋₃ acyl, —SR⁶⁰, —S(O)₂OH, —S(O)₂R⁶⁰, —S(O)R⁶⁰, —C(S)R⁶⁰, —C(O)O⁻, —C(S)OR⁶⁰, —NR⁶⁰C(O)NR⁶¹R⁶², —NR⁶⁰C(S)NR⁶¹R⁶², and —C(NR⁶⁰)NR⁶¹R⁶², C₃₋₈ cycloalkyl, and substituted C₃₋₈ cycloalkyl, wherein R⁶⁰, R⁶¹, and R⁶² are independently chosen from hydrogen and C₁₋₄ alkyl.

In certain embodiments, each substituent group can independently be chosen from halogen, —NO₂, —OH, —COOH, —NH₂, —CN, —CF₃, —OCF₃, C₁₋₈ alkyl, substituted C₁₋₈ alkyl, C₁₋₈ alkoxy, and substituted C₁₋₈ alkoxy.

“Sustained release” refers to release of a therapeutic amount of a drug, a prodrug, or an active metabolite of a prodrug over a period of time that is longer than that of a conventional formulation of the drug, e.g. an immediate release formulation of the compound. For oral formulations, the term “sustained release” typically means release of the compound within the gastrointestinal tract lumen over a time period from about 2 to about 30 hours, and in certain embodiments, over a time period from about 4 to about 24 hours. Sustained release formulations achieve therapeutically effective concentrations of the drug in the systemic circulation over a prolonged period of time relative to that achieved by oral administration of an immediate release formulation of the drug. “Delayed release” refers to release of a drug, a prodrug, or an active metabolite of a prodrug into the gastrointestinal lumen after a delayed time period, for example a delay of about 1 to about 12 hours, relative to that achieved by oral administration of an immediate release formulation of the drug.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease, disorder, or at least one of the clinical symptoms of a disease or disorder. “Treating” or “treatment” also refers to inhibiting the disease, disorder, or at least one of the clinical symptoms of a disease or disorder, either physically, (e.g., stabilization of a discernible symptom), physiologically, (e.g., stabilization of a physical parameter), or both, and to inhibiting at least one physical parameter which may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The “therapeutically effective amount” can vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate therapeutically effective amount in any given instance may be ascertained by those skilled in the art or capable of determination by routine experimentation.

“Therapeutically effective dose” refers to a dose of a drug, prodrug or active metabolite of a prodrug that provides effective treatment of a disease or disorder in a patient. A therapeutically effective dose may vary from compound to compound and from patient to patient, and may depend upon factors such as the condition of the patient and the route of delivery. A therapeutically effective dose may be determined in accordance with routine pharmacological procedures known to those skilled in the art.

Reference is now made in detail to embodiments of the present disclosure. The disclosed embodiments are not intended to be limiting of the claims. To the contrary, the claims are intended to cover alternatives, modifications, and equivalents.

### GABA_{B} Agonists

GABA_{B} receptor agonist are compounds that elicit a positive effect in a GABA_{B} agonist functional assay such as the cAMP, Ca²⁺, and electrophysiology in vitro assays, the hypothermia animal model, or in any other accepted functional assay for determining GABA_{B} receptor agonist activity known in the art. For example, a GABA_{B} agonist can be identified using the in vitro and/or in vivo assays described in Examples 1-4.

Full GABA_{B} receptor agonists bind to the binding site of endogenous GABA_{B} receptor agonist and display full efficacy. Partial GABA_{B} agonists also bind at the GABA_{B} receptor at the endogenous agonist binding site and activate the receptor but exhibit only partial efficacy relative to a full agonist. Partial agonists can also be considered ligands that exhibit both agonistic and antagonistic effects, e.g., the presence of a partial agonist will reduce the receptor activation of a full agonist. The capacity for a compound to function as a partial GABA_{B} receptor agonist can be assessed by determining the maximal response in a GABA_{B} receptor agonist activity assay. A GABA_{B} receptor agonist will demonstrate a response equal to or nearly equal to that of a known reference GABA_{B} receptor agonist such as GABA or R-baclofen. A partial agonist will demonstrate a response less than that of a full response. In certain embodiments, a GABA_{B} agonist is a compound of Formula (IV):

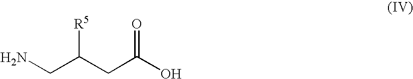

where R⁵ is chosen from substituted aryl, heteroaryl and substituted heteroaryl. In certain embodiments of a compound of Formula (IV), R⁵ is chosen from 4-chlorophenyl, (3R)-4-chlorophenyl, 2-chlorophenyl, 4-fluorophenyl, thien-2-yl; 5-chlorothien-2-yl, 5-bromothien-2-yl, 5-methylthien-2-yl, and 2-imidazolyl. In certain embodiments of a compound of Formula (IV), R⁵ is chosen from 4-chlorophenyl, (3R)-4-chlorophenyl, 2-chlorophenyl, 4-fluorophenyl. In certain embodiments, a a compound of Formula (IV) wherein R⁵ is 4-chlorophenyl is R-baclofen, i.e., (R)-4-amino-3-(4-chlorophenyl)butanoic acid

### Prodrugs of GABA_{B} Agonists

Reducing the rate of metabolism of a drug in the gastrointestinal tract and/or enhancing the rate by which a drug is absorbed from the gastrointestinal tract may enhance the oral bioavailability of a drug. An orally administered drug will pass through the gastrointestinal system in about 11 to 31 hours. In general, an orally ingested drug resides about 1 to 6 hours in the stomach, about 2 to 7 hours in the small intestine, and about 8 to 18 hours in the colon. The oral bioavailability of a particular drug will depend on a number of factors including the residence time in a particular region of the gastrointestinal tract, the rate the drug is metabolized within the gastrointestinal tract, the rate at which a drug is metabolized in the systemic circulation, and the rate by which the compound is absorbed from a particular region or regions of the gastrointestinal tract, which include passive and active transport mechanisms. Several methods have been developed to achieve these objectives, including drug modification, incorporating the drug or modified drug in a controlled release dosage form, and/or by co-administering adjuvants, which can be incorporated in the dosage form containing the active compound.

Examples of prodrugs of GABA_{B} agonists that provide a high oral bioavailability of the corresponding GABA_{B} agonist include compounds of Formulae (I)-(III). Prodrugs are compounds in which a promoiety is typically covalently bonded to a drug. Following absorption from the gastrointestinal tract, the promoiety is cleaved to release the drug into the systemic circulation. While in the gastrointestinal tract, the promoiety can protect the drug from the harsh chemical environment, and can also facilitate absorption. Promoieties can be designed, for example, to enhance passive absorption, e.g., lipophilic promoieties, and/or to enhance absorption via active transport mechanisms, e.g., substrate promoieties. In particular, active transporters differentially expressed in regions of the gastrointestinal tract may be preferentially targeted to enhance absorption. For example, a prodrug of a GABA_{B} agonist may incorporate a promoiety that is a substrate of the PEPT1 transporter expressed in the small intestine. Zerangue et al., U.S. Pat. No. 6,955,888 and US 2005/0214853, each of which is incorporated by reference herein in its entirety, disclose methodologies for screening drugs, conjugates or conjugate moieties, linked or linkable to drugs, for their capacity to be transported as substrates via the PEPT1 and PEPT2 transporters, which are known to be expressed in the human small intestine. Zerangue et al., US 2003/0158254 also disclose several transporters expressed in the human colon including the sodium dependent multi-vitamin transporter (SMVT) and monocarboxylate transporters MCT1 and MCT4, and methods of identifying agents, or conjugate moieties that are transporter substrates, and agents, conjugates, and conjugate moieties that may be screened for substrate activity. Zerangue et al. further disclose compounds that may be screened and are variants of known transporter substrates such as bile salts or acids, steroids, ecosanoids, or natural toxins or analogs thereof, as well as the linkage of drugs to conjugate moieties.

Examples of prodrugs of GABA_{B} agonists capable of providing an increased oral bioavailability of the corresponding GABA_{B} agonist are disclosed in Gallop et al., U.S. Pat. No. 7,109,239 and US 2008-0096960, each of which is incorporated by reference herein in its entirety.

In certain embodiments, prodrugs of GABA_{B} agonists may be chosen from any of the genera or species of compounds of Formula (I) as disclosed in Gallop et al., U.S. Pat. No. 7,109,239:

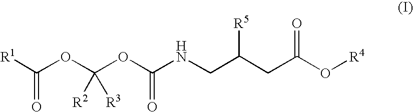

or a pharmaceutically acceptable salt thereof, wherein:

R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, cycloheteroalkyl, and substituted cycloheteroalkyl ring;

R⁴ is chosen from hydrogen, alkyl, substituted alkyl, aryl, substituted aryl, arylalkyl, substituted arylalkyl, aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, and trialkylsilyl; and

R⁵ is chosen from substituted aryl, heteroaryl, and substituted heteroaryl. In certain embodiments of a compound of Formula (I), each substituent is independently chosen from halogen, —OH, —CN, —CF₃, —C(O)NH₂, —COOR¹⁰, and NR¹⁰₂ wherein each R¹⁰ is independently chosen from hydrogen and C₁₋₃ alkyl.

In certain embodiments of a compound of Formula (I), R¹ is chosen from methyl, ethyl, n-propyl, isopropyl, n-butyl, isobutyl, sec-butyl, tert-butyl, n-pentyl, isopentyl, sec-pentyl, neopentyl, 1,1-diethoxyethyl, phenyl, cyclohexyl, 2-pyridyl, 3-pyridyl, and 4-pyridyl; R² is chosen from hydrogen, methyl, ethyl, n-propyl, isopropyl, n-butyl, isobutyl, sec-butyl, phenyl, and cyclohexyl; R³ is hydrogen; and R⁴ is hydrogen.

In certain embodiments of a compound of Formula (I), R¹ is chosen from methyl, ethyl, n-propyl, isopropyl, n-butyl, isobutyl, sec-butyl, tert-butyl, phenyl, cyclohexyl, and 3-pyridyl; R² is hydrogen; R³ is hydrogen; and R⁴ is hydrogen.

In certain embodiments of a compound of Formula (I), R¹ is chosen from methyl, ethyl, n-propyl, isopropyl, n-butyl, isobutyl, sec-butyl, tert-butyl, phenyl, and cyclohexyl; R² is chosen from methyl, n-propyl, and isopropyl; R³ is hydrogen; and R⁴ is hydrogen.

In certain embodiments of a compound of Formula (I), R¹ is chosen from methyl, ethyl, n-propyl, isopropyl, n-butyl, isobutyl, sec-butyl, tert-butyl, phenyl, and cyclohexyl; R² is isopropyl; R³ is hydrogen; and R⁴ is hydrogen.

In certain embodiments of a compound of Formula (I), R¹ is isopropyl; R² is isopropyl; R³ is hydrogen; and R⁴ is hydrogen.

In certain embodiments of a compound of Formula (I), R⁵ is chosen from 4-chlorophenyl, (3R)-4-chlorophenyl, 2-chlorophenyl, 4-fluorophenyl, thien-2-yl; 5-chlorothien-2-yl, 5-bromothien-2-yl, 5-methylthien-2-yl, and 2-imidazolyl. In certain embodiments, R⁵ is chosen from 4-chlorophenyl, (3R)-4-chlorophenyl, 2-chlorophenyl, and 4-fluorophenyl.

In certain embodiments of a compound of Formula (I), R⁵ is 4-chlorophenyl and the carbon to which R⁵ is bonded is of the R-configuration.

In certain embodiments, prodrugs of GABA_{B} agonists may be chosen from any of the genera or species of compounds of Formula (II) as disclosed in Gallop et al., U.S. Pat. No. 7,109,239:

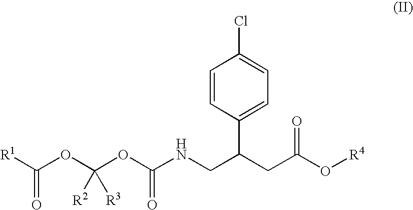

or a pharmaceutically acceptable salt thereof, wherein:

R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, cycloheteroalkyl, and substituted cycloheteroalkyl ring; and

R⁴ is chosen from hydrogen, alkyl, substituted alkyl, aryl, substituted aryl, arylalkyl, substituted arylalkyl, aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, and trialkylsilyl.

In certain embodiments of a compound of Formula (II), each substituent is independently chosen from halogen, —OH, —CN, —CF₃, —C(O)NH₂, —COOR¹⁰, and —NR¹⁰₂ wherein each R¹⁰ is independently chosen from hydrogen and C₁₋₃ alkyl.

In certain embodiments of a compound of Formula (II), R¹ is chosen from methyl, ethyl, n-propyl, isopropyl, n-butyl, isobutyl, sec-butyl, tert-butyl, n-pentyl, isopentyl, sec-pentyl, neopentyl, 1,1-diethoxyethyl, phenyl, cyclohexyl, 2-pyridyl, 3-pyridyl, and 4-pyridyl; R² is chosen from hydrogen, methyl, ethyl, n-propyl, isopropyl, n-butyl, isobutyl, sec-butyl, phenyl, and cyclohexyl; R³ is hydrogen; and R⁴ is hydrogen.

In certain embodiments of a compound of Formula (II), R¹ is chosen from methyl, ethyl, n-propyl, isopropyl, n-butyl, isobutyl, sec-butyl, tert-butyl, phenyl, cyclohexyl, and 3-pyridyl; R² is hydrogen; R³ is hydrogen; and R⁴ is hydrogen.

In certain embodiments of a compound of Formula (II), R¹ is chosen from methyl, ethyl, n-propyl, isopropyl, n-butyl, isobutyl, sec-butyl, tert-butyl, phenyl, and cyclohexyl; R² is chosen from methyl, n-propyl, and isopropyl; R³ is hydrogen; and R⁴ is hydrogen.

In certain embodiments of a compound of Formula (II), R¹ is chosen from methyl, ethyl, n-propyl, isopropyl, n-butyl, isobutyl, sec-butyl, tert-butyl, phenyl, and cyclohexyl; R² is isopropyl; R³ is hydrogen; and R⁴ is hydrogen.

In certain embodiments of a compound of Formula (II), R¹ is isopropyl; R² is isopropyl; R³ is hydrogen; and R⁴ is hydrogen.

In certain embodiments, prodrugs of GABA_{B} agonists may be chosen from any of the genera or species of compounds of Formula (III) as disclosed in Gallop et al., U.S. Pat. No. 7,109,239:

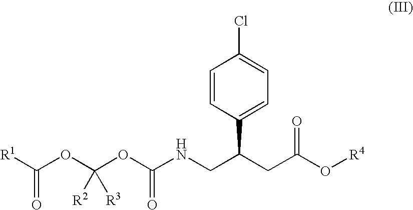

or a pharmaceutically acceptable salt thereof, wherein:

R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, cycloheteroalkyl, and substituted cycloheteroalkyl ring; and

R⁴ is chosen from hydrogen, alkyl, substituted alkyl, aryl, substituted aryl, arylalkyl, substituted arylalkyl, aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, and trialkylsilyl.

In certain embodiments of a compound of Formula (III), each substituent is independently chosen from halogen, —OH, —CN, —CF₃, —C(O)NH₂, —COOR¹⁰, and —NR¹⁰₂ wherein each R¹⁰ is independently chosen from hydrogen and C₁₋₃ alkyl.

In certain embodiments of a compound of Formula (III), R¹ is chosen from methyl, ethyl, n-propyl, isopropyl, n-butyl, isobutyl, sec-butyl, tert-butyl, n-pentyl, isopentyl, sec-pentyl, neopentyl, 1,1-diethoxyethyl, phenyl, cyclohexyl, 2-pyridyl, 3-pyridyl, and 4-pyridyl; R² is chosen from hydrogen, methyl, ethyl, n-propyl, isopropyl, n-butyl, isobutyl, sec-butyl, phenyl, and cyclohexyl; R³ is hydrogen; and R⁴ is hydrogen.

In certain embodiments of a compound of Formula (III), R¹ is chosen from methyl, ethyl, n-propyl, isopropyl, n-butyl, isobutyl, sec-butyl, tert-butyl, phenyl, cyclohexyl, and 3-pyridyl; R² is hydrogen; R³ is hydrogen; and R⁴ is hydrogen.

In certain embodiments of a compound of Formula (III), R¹ is chosen from methyl, ethyl, n-propyl, isopropyl, n-butyl, isobutyl, sec-butyl, tert-butyl, phenyl, and cyclohexyl; R² is chosen from methyl, n-propyl, and isopropyl; R³ is hydrogen; and R⁴ is hydrogen.

In certain embodiments of a compound of Formula (III), R¹ is chosen from methyl, ethyl, n-propyl, isopropyl, n-butyl, isobutyl, sec-butyl, tert-butyl, phenyl, and cyclohexyl; R² is isopropyl; R³ is hydrogen; and R⁴ is hydrogen.

In certain embodiments of a compound of Formula (III), R¹ is isopropyl; R² is isopropyl; R³ is hydrogen; and R⁴ is hydrogen.

In certain embodiments of a compound of Formula (III), the compound is (3R)-4-{[(1S)-2-methyl-1-(2-methylpropanoyloxy)propoxy]carbonylamino}-3-(4-chorophenyl)butanoic acid.

Methods of synthesizing colonically absorbable prodrugs of GABA_{B} agonists of Formulae (I)-(III) are disclosed, for example, in Gallop et al., U.S. Pat. No. 7,109,239 and U.S. Pat. No. 7,227,028, and Raillard et al., U.S. Provisional Application Nos. 61/087,056 and 61/087,038, both filed Aug. 7, 2008, each of which is incorporated by reference herein in its entirety.

In certain embodiments, prodrugs of GABA_{B} agonists of Formulae (I)-(III) provide a colonic bioavailability of the corresponding GABA_{B} agonist that is at least 2 times greater than the colonic bioavailability of the same GABA_{B} agonist when colonically administered in an equivalent dosage form. In certain embodiments, prodrugs of GABA_{B} agonists provide a colonic bioavailability of the corresponding GABA_{B} agonist that is at least 2 times greater than the colonic bioavailability of the GABA_{B} agonist provided by the same GABA_{B} agonist when colonically administered to a patient as a uniform liquid immediate release formulation.

### Pharmaceutical Compositions

Prodrugs of GABA_{B} agonists provided by the present disclosure may be formulated into pharmaceutical compositions for use in oral dosage forms to be administered to patients.

Pharmaceutical compositions comprise at least one prodrug of a GABA_{B} agonist and at least one pharmaceutically acceptable vehicle. A pharmaceutical composition may comprise a therapeutically effective dose of at least one prodrug of a GABA_{B} agonist and at least one pharmaceutically acceptable vehicle. Pharmaceutically acceptable vehicles include diluents, adjuvants, excipients, and carriers. Pharmaceutical compositions may be produced using methods known in the art. Pharmaceutical compositions may take any form appropriate for oral delivery such as solutions, suspensions, emulsions, tablets, pills, pellets, granules, capsules, capsules containing liquids, powders, and the like. Pharmaceutical compositions provided by the present disclosure may be formulated so as to provide immediate, sustained, or delayed release of a prodrug of a GABA_{B} agonist or metabolite thereof after administration to a patient by employing procedures known in the art.

Pharmaceutical compositions may include an adjuvant that facilitates absorption of a prodrug of a GABA_{B} agonist through the gastrointestinal epithelia. Such enhancers may, for example, open the tight-junctions in the gastrointestinal tract or modify the effect of cellular components, such as p-glycoprotein and the like. Suitable enhancers include alkali metal salts of salicylic acid, such as sodium salicylate, caprylic, or capric acid, such as sodium caprylate or sodium caprate, sodium deoxycholate, and the like. Other adjuvants that enhance permeability of cellular membranes include resorcinol, surfactants, polyethylene glycol, and bile acids. Adjuvants may also reduce enzymatic degradation of a compound of a prodrug of a GABA_{B} agonist. Microencapsulation using protenoid microspheres, liposomes, or polysaccharides may also be effective in reducing enzymatic degradation of administered compounds.

Prodrugs of GABA_{B} agonists provided by the present disclosure may be formulated in unit oral dosage forms. Unit oral dosage forms refer to physically discrete units suitable for dosing to a patient undergoing treatment, with each unit containing a predetermined quantity of a prodrug of a GABA_{B} agonist. Oral dosage forms comprising at least one prodrug of a GABA_{B} agonist may be administered to patients as a dose, with each dose comprising one or more oral dosage forms. A dose may be administered once a day, twice a day, or more than twice a day, such as three or four times per day. A dose may be administered at a single point in time or during a time interval. Oral dosage forms comprising at least one prodrug of a GABA_{B} agonist may be administered alone or in combination with other drugs for treating the same or different disease, and may continue as long as required for effective treatment of the disease. Oral dosage forms comprising a prodrug of a GABA_{B} agonist may provide a concentration of the corresponding GABA_{B} agonist in the plasma, blood, or tissue of a patient over time, following oral administration of the dosage form to the patient. The GABA_{B} agonist concentration profile may exhibit an AUC that is proportional to the dose of the prodrug of the GABA_{B} agonist.

A dose comprises an amount of a prodrug of a GABA_{B} agonist calculated to produce an intended therapeutic effect. An appropriate amount of a prodrug of the corresponding GABA_{B} agonist to produce an intended therapeutic effect will depend, in part, on the oral bioavailability of the prodrug of the GABA_{B} agonist and/or metabolite thereof, by the pharmacokinetics of the prodrug, and/or by the properties of the dosage form used to administer the prodrug. A therapeutically effective dose of a prodrug of a GABA_{B} agonist for treating neuropathic or musculoskeletal pain may comprise about 1 mg-equivalents to about 200 mg-equivalents of the corresponding GABA_{B} agonist, about 1 mg-equivalents to about 100 mg-equivalents of the corresponding GABA_{B} agonist, and in certain embodiments, about 1 mg-equivalents to about 50 mg-equivalents of the corresponding GABA_{B} agonist. For example, doses of baclofen in the range of about 50 mg per day to about 60 mg per day are effective in treating trigeminal neuralgia (Sidebottom and Maxwell, J Clin Pharm Ther 1995, 20, 31-35; Green and Selman, Headache 1991, 31, 588-92; and Fromm, Clin Neuropharmacol 1990, 8, 143-51) and doses of baclofen in the range of about 30 mg per day to about 80 mg per day have been shown effective in the treatment of low back pain (Dapas et al., Spine 1985, 10(4), 345-9). In certain embodiments, a therapeutically effective dose of a prodrug of a GABA_{B} agonist prodrug for treating neuropathic or musculoskeletal pain is less than a dose that causes moderate sedation and/or impaired motor coordination in a patient.

In certain embodiments, a therapeutically effective dose of a GABA_{B} agonist prodrug or a pharmaceutically acceptable salt thereof comprises about 1 mg-equivalent of the corresponding GABA_{B} agonist to about 200 mg-equivalent of the corresponding GABA_{B} agonist, about 1 mg-equivalent of the corresponding GABA_{B} agonist to about 100 mg-equivalent of the corresponding GABA_{B} agonist, and in certain embodiments, about 1 mg-equivalent of the corresponding GABA_{B} agonist to about 50 mg-equivalent of the corresponding GABA_{B} agonist. In certain embodiments, a therapeutically effective dose of a prodrug of a GABA_{B} agonist comprises about 1 mg-equivalents/day to about 1,000 mg-equivalents/day of the corresponding GABA_{B} agonist, about 10 mg-equivalents/day to about 500 mg-equivalents/day of the corresponding GABA_{B} agonist, and in certain embodiments, about 20 mg-equivalents/day to about 250 mg-equivalents/day of the corresponding GABA_{B} agonist.

In certain embodiments wherein the GABA_{B} agonist prodrug is (a compound of Formula (II), Formula (III), or a pharmaceutically acceptable salt thereof, a therapeutically effective dose comprises about 1 mg-equivalent of (R)-baclofen to about 200 mg-equivalent of (R)-baclofen, about 1 mg-equivalent of (R)-baclofen to about 100 mg-equivalent of (R)-baclofen, and in certain embodiments, about 1 mg-equivalent of (R)-baclofen to about 50 mg-equivalent of (R)-baclofen. In certain embodiments, wherein the GABA_{B} agonist prodrug is a compound of Formula (II), Formula (III), or a pharmaceutically acceptable salt thereof, a therapeutically effective dose comprises about 1 mg-equivalents/day to about 500 mg-equivalents/day of the (R)-baclofen, about 10 mg-equivalents/day to about 300 mg-equivalents/day of (R)-baclofen, and in certain embodiments, about 20 mg-equivalents/day to about 100 mg-equivalents/day of (R)-baclofen.

In certain embodiments, wherein the GABA_{B} agonist prodrug is (3R)-4-{[(1S)-2-methyl-1-(2-methylpropanoyloxy)propoxy]carbonylamino}-3-(4-chlorophenyl)butanoic acid, or a pharmaceutically acceptable salt thereof, a therapeutically effective dose comprises about 1 mg-equivalent of (R)-baclofen to about 200 mg-equivalent of (R)-baclofen, about 1 mg-equivalent of (R)-baclofen to about 100 mg-equivalent of (R)-baclofen, and in certain embodiments, about 1 mg-equivalent of (R)-baclofen to about 50 mg-equivalent of (R)-baclofen. In certain embodiments, wherein the GABA_{B} agonist prodrug is (3R)-4-{[(1S)-2-methyl-1-(2-methylpropanoyloxy)propoxy]carbonylamino}-3-(4-chlorophenyl)butanoic acid, or a pharmaceutically acceptable salt thereof, a therapeutically effective dose comprises about 1 mg-equivalents/day to about 500 mg-equivalents/day of the (R)-baclofen, about 10 mg-equivalents/day to about 300 mg-equivalents/day of (R)-baclofen, and in certain embodiments, about 20 mg-equivalents/day to about 100 mg-equivalents/day of (R)-baclofen.

In certain embodiments, a therapeutically effective dose of a prodrug of a GABA_{B} agonist provides a blood concentration of the corresponding GABA_{B} agonist from about 10 ng/mL to about 500 ng/mL, in certain embodiments from about 20 ng/mL to about 400 ng/mL, and in certain embodiments from about 40 ng/mL to about 200 ng/mL for a continuous period of time following oral administration of a dosage form comprising the corresponding prodrug of a GABA_{B} agonist to a patient. In certain embodiments, a therapeutically effective dose of a prodrug of a GABA_{B} agonist provides a blood concentration of the corresponding GABA_{B} agonist that is therapeutically effective for treating neuropathic or musculoskeletal pain in a patient, and that is less than a concentration of the corresponding GABA_{B} agonist effective in causing moderate sedation and/or impaired motor coordination in the patient, for example, less than about 800 ng/mL, less than about 400 ng/mL, or less than about 200 ng/mL.

In certain embodiments, wherein the GABA_{B} agonist prodrug is a compound of Formula (II), Formula (III), or a pharmaceutically acceptable salt thereof, a therapeutically effective dose comprises about 1 mg-equivalent of (R)-baclofen to about 200 mg-equivalent of (R)-baclofen, about 1 mg-equivalent of (R)-baclofen to about 100 mg-equivalent of (R)-baclofen, and in certain embodiments, about 1 mg-equivalent of (R)-baclofen to about 50 mg-equivalent of (R)-baclofen. In certain embodiments, wherein the GABA_{B} agonist prodrug is a compound of Formula (II), Formula (III), or a pharmaceutically acceptable salt thereof, a therapeutically effective dose comprises about 1 mg-equivalents/day to about 500 mg-equivalents/day of the (R)-baclofen, about 10 mg-equivalents/day to about 300 mg-equivalents/day of (R)-baclofen, and in certain embodiments, about 20 mg-equivalents/day to about 100 mg-equivalents/day of (R)-baclofen.

In certain embodiments wherein the GABA_{B} agonist prodrug is a compound of Formula (II), Formula (III), or a pharmaceutically acceptable salt thereof, a therapeutically effective dose provides a blood concentration of (R)-baclofen from about 10 ng/mL to about 500 ng/mL, in certain embodiments from about 20 ng/mL to about 400 ng/mL, and in certain embodiments from about 40 ng/mL to about 200 ng/mL for a continuous period of time following oral administration of a dosage form comprising the compound of Formula (II), Formula (III), or a pharmaceutically acceptable salt thereof to a patient. In certain embodiments wherein the GABA_{B} agonist prodrug is a compound of Formula (II), Formula (III), or a pharmaceutically acceptable salt thereof, a therapeutically effective dose provides a blood concentration of (R)-baclofen that is therapeutically effective for treating neuropathic or musculoskeletal pain in a patient, and that is less than a concentration of (R)-baclofen effective in causing moderate sedation and/or impaired motor coordination in the patient, for example, less than about 400 ng/mL, less than about 200 ng/mL, or less than about 100 ng/mL.

In certain embodiments wherein the GABA_{B} agonist prodrug is a compound of Formula (II), Formula (III), or a pharmaceutically acceptable salt thereof, a therapeutically effective dose provides a blood concentration of (R)-baclofen from about 10 ng/mL to about 500 ng/mL, in certain embodiments from about 20 ng/mL to about 400 ng/mL, and in certain embodiments from about 40 ng/mL to about 200 ng/mL for a continuous period of time following oral administration of a dosage form comprising (3R)-4-{[(1S)-2-methyl-1-(2-methylpropanoyloxy)propoxy]carbonylamino}-3-(4-chlorophenyl)butanoic acid, or a pharmaceutically acceptable salt thereof to a patient. In certain embodiments wherein the GABA_{B} agonist prodrug is (3R)-4-{[(1S)-2-methyl-1-(2-methylpropanoyloxy)propoxy]carbonylamino)-3-(4-chlorophenyl}butanoic acid, or a pharmaceutically acceptable salt thereof, a therapeutically effective dose provides a blood concentration of (R)-baclofen that is therapeutically effective for treating neuropathic or musculoskeletal pain in a patient, and that is less than a concentration of (R)-baclofen effective in causing moderate sedation and/or impaired motor coordination in the patient, for example, less than about 400 ng/mL, less than about 200 ng/mL, or less than about 100 ng/mL.

In certain embodiments wherein the GABA_{B} agonist prodrug is a compound of Formula (II), Formula (III), or a pharmaceutically acceptable salt thereof, administration of an oral dosage form comprising (3R)-4-{[(1S)-2-methyl-1-(2-methylpropanoyloxy)propoxy]carbonylamino}-3-(4-chlorophenyl)butanoic acid or a pharmaceutically acceptable salt thereof provides a maximum plasma concentration (Cₘₐₓ) of less than 200 ng/mL of (R)-baclofen and a total plasma (R)-baclofen exposure of at least 1,500 ng-hr/mL (AUC₀₋₂₄). In certain embodiments wherein the GABA_{B} agonist prodrug is a compound of Formula (II), Formula (III), or a pharmaceutically acceptable salt thereof, administration of an oral dosage form comprising (3R)-4-{[(1S)-2-methyl-1-(2-methylpropanoyloxy)propoxy]carbonylamino}-3-(4-chlorophenyl)butanoic acid or a pharmaceutically acceptable salt thereof provides a maximum plasma concentration (Cₘₐₓ) of less than 150 ng/mL of (R)-baclofen and a total plasma (R)-baclofen exposure of at least 1,000 ng-hr/mL (AUC₀₋₂₄).

In certain embodiments wherein the GABA_{B} agonist prodrug is (3R)-4-{[(1S)-2-methyl-1-(2-methylpropanoyloxy)propoxy]carbonylamino}-3-(4-chlorophenyl)butanoic acid or a pharmaceutically acceptable salt thereof administration of an oral dosage form comprising (3R)-4-{[(1S)-2-methyl-1-(2-methylpropanoyloxy)propoxy]carbonylamino}-3-(4-chlorophenyl)butanoic acid or a pharmaceutically acceptable salt thereof provides a maximum plasma concentration (Cₘₐₓ) of less than 200 ng/mL of (R)-baclofen and a total plasma (R)-baclofen exposure of at least 1,500 ng-hr/mL (AUC₀₋₂₄). In certain embodiments wherein the GABA_{B} agonist prodrug is (3R)-4-{[(1S)-2-methyl-1-(2-methylpropanoyloxy)propoxy]carbonylamino}-3-(4-chlorophenyl)butanoic acid or a pharmaceutically acceptable salt thereof administration of an oral dosage form comprising (3R)-4-{[(1S)-2-methyl-1-(2-methylpropanoyloxy)propoxy]carbonylamino}-3-(4-chlorophenyl)butanoic acid or a pharmaceutically acceptable salt thereof provides a maximum plasma concentration (Cₘₐₓ) of less than 150 ng/mL of (R)-baclofen and a total plasma (R)-baclofen exposure of at least 1,000 ng-hr/mL (AUC₀₋₂₄).

Oral dosage forms comprising a prodrug of a GABA_{B} agonist may have immediate release or controlled release characteristics. Immediate release oral dosage forms release the prodrug from the dosage form within about 30 minutes following ingestion. In certain embodiments, an oral dosage form provided by the present disclosure may be a controlled release dosage form. Controlled delivery technologies may improve the absorption of a drug in a particular region or regions of the gastrointestinal tract. Controlled drug delivery systems may be designed to deliver a drug in such a way that the drug level is maintained within a therapeutically effective blood concentration range for a period as long as the system continues to deliver the drug at a particular rate. Controlled drug delivery may produce substantially constant blood levels of a drug as compared to fluctuations observed with immediate release dosage forms. For some diseases maintaining a controlled concentration of a GABA_{B} agonist in the blood or in a tissue throughout the course of therapy is desirable. Immediate release dosage forms may cause blood levels to peak above the level required to elicit the desired response, which may cause or exacerbate side effects. Controlled drug delivery may result in optimum therapy, reduce the frequency of dosing, and reduce the occurrence, frequency, and/or severity of side effects. Examples of controlled release dosage forms include dissolution controlled systems, diffusion controlled systems, ion exchange resins, osmotically controlled systems, erodable matrix systems, pH independent formulations, gastric retention systems, and the like.

The appropriate oral dosage form for a particular prodrug of a GABA_{B} agonist may depend, at least in part, on the gastrointestinal absorption properties of the prodrug, the stability of the prodrug in the gastrointestinal tract, the pharmacokinetics of the prodrug of a GABA_{B} agonist, the pharmacokinetics of the corresponding GABA_{B} agonist, and the intended therapeutic profile of the corresponding GABA_{B} agonist. An appropriate controlled release oral dosage form may be selected for a particular prodrug of a GABA_{B} agonist. For example, gastric retention oral dosage forms may be appropriate for prodrugs of GABA_{B} agonists absorbed primarily from the upper gastrointestinal tract, and sustained release oral dosage forms may be appropriate for prodrugs GABA_{B} agonists absorbed primarily form the lower gastrointestinal tract.

Gastric retention dosage forms, i.e., dosage forms designed to be retained in the stomach for a prolonged period of time can increase the bioavailability of drugs that are most readily absorbed from the upper gastrointestinal tract. The residence time of a conventional dosage form in the stomach is 1 to 3 hours. After transiting the stomach, there is approximately a 3 to 5 hour window of bioavailability before the dosage form reaches the colon. However, if the dosage form is retained in the stomach, the drug can be released before it reaches the small intestine and will enter the intestine in solution in a state in which it can be more readily absorbed. Another use of gastric retention dosage forms is to improve the bioavailability of a drug that is unstable to the basic conditions of the intestine. To enhance drug absorption from the upper gastrointestinal tract, several gastric retention dosage forms have been developed including hydrogels, buoyant matrices, polymer sheets, microcellular foams, swellable dosage forms, Bioadhesive polymers, ion exchange resins, and polymer matrices.

Prodrugs of GABA_{B} agonists may be practiced with a number of different dosage forms adapted to provide sustained release of a prodrug of a GABA_{B} agonist upon oral administration. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems.

Sustained release oral dosage forms may be in any appropriate form suitable for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, and the like.

Sustained release oral dosage forms may release a prodrug of a GABA_{B} agonist from the dosage form to facilitate the ability of the prodrug and/or GABA_{B} agonist metabolite to be absorbed from an appropriate region of the gastrointestinal tract, for example, in the small intestine, or in the colon. In certain embodiments, sustained release oral dosage forms may release a prodrug of a GABA_{B} agonist from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms may release a prodrug of a GABA_{B} agonist from the dosage form in a delivery pattern in which from about 0 wt % to about 20 wt % of the prodrug is released in about 0 to about 4 hours, about 20 wt % to about 50 wt % of the prodrug is released in about 0 to about 8 hours, about 55 wt % to about 85 wt % of the prodrug is released in about 0 to about 14 hours, and about 80 wt % to about 100 wt % of the prodrug is released in about 0 to about 24 hours. In certain embodiments, sustained release oral dosage forms may release a prodrug of a GABA_{B} agonist from the dosage form in a delivery pattern in which from about 0 wt % to about 20 wt % of the prodrug is released in about 0 to about 4 hours, about 20 wt % to about 50 wt % of the prodrug of a GABA_{B} agonist is released in about 0 to about 8 hours, about 55 wt % to about 85 wt % of the prodrug is released in about 0 to about 14 hours, and about 80 wt % to about 100 wt % of the prodrug is released in about 0 to about 20 hours. In certain embodiments, sustained release oral dosage forms may release a prodrug of a GABA_{B} agonist from the dosage form in a delivery pattern in which from about 0 wt % to about 20 wt % of the prodrug is released in about 0 to about 2 hours, about 20 wt % to about 50 wt % of the prodrug is released in about 0 to about 4 hours, about 55 wt % to about 85 wt % of the prodrug is released in about 0 to about 7 hours, and about 80 wt % to about 100 wt % of the prodrug is released in about 0 to about 8 hours.

Regardless of the specific form of oral dosage form used, a prodrug of a GABA_{B} agonist may be released from the orally administered dosage form over a sufficient period of time to provide prolonged therapeutic concentrations of the corresponding GABA_{B} agonist in the blood of a patient. Following oral administration, dosage forms comprising a prodrug of a GABA_{B} agonist may provide a therapeutically effective concentration of the corresponding GABA_{B} agonist in the blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous period of time during which a therapeutically effective blood concentration of a GABA_{B} agonist is maintained may begin shortly after oral administration or following a time interval.

In certain embodiments, it may be desirable that the blood concentration of a GABA_{B} agonist be maintained at a level between a concentration that causes moderate sedation and/or impaired motor coordination in the patient and a minimum therapeutically effective concentration for treating neuropathic or musculoskeletal pain for a continuous period of time. The blood concentration of a GABA_{B} agonist that causes moderate sedation or impaired motor coordination in a patient can vary depending on the individual patient. In certain embodiments, a minimum therapeutically effective blood GABA_{B} agonist concentration will be about 2 ng/mL, about 5 ng/mL, about 10 ng/mL, about 20 ng/mL, about 30 ng/mL, about 40 ng/mL, about 50 ng/mL, or about 60 ng/mL. In certain embodiments, a therapeutically effective blood concentration of a GABA_{B} agonist for treating neuropathic or musculoskeletal pain is from about 1 ng/mL to less than about 400 ng/mL, and in certain embodiments from about 10 ng/mL to less than about 200 ng/mL. In certain embodiments, a therapeutically effective blood concentration of a GABA_{B} agonist for treating neuropathic pain is from about 10 ng/mL to less than a concentration that causes moderate sedation and/or impaired motor coordination. In certain embodiments, a therapeutically effective blood concentration of a GABA_{B} agonist for treating neuropathic or musculoskeletal pain is from about 2 ng/mL to about 400 ng/mL. In certain embodiments, methods provided by the present disclosure provide a blood GABA_{B} agonist concentration that, following oral administration to a patient, does not produce sedation and/or impaired motor coordination in the patient. In certain embodiments, methods provided by the present disclosure provide a blood GABA_{B} agonist concentration that, following oral administration to a patient, produces moderate sedation in a patient.

Prodrugs of GABA_{B} agonists may be absorbed from the gastrointestinal tract and enter the systemic circulation intact. In certain embodiments, a prodrug of a GABA_{B} agonist exhibits an oral bioavailability of the prodrug greater than about 40% that of an equivalent intravenous dose of the prodrug, greater than about 60%, and in certain embodiments greater than about 80%. In certain of the foregoing embodiments, a prodrug of a GABA_{B} agonist exhibits an oral bioavailability of the corresponding GABA_{B} agonist greater than about 10% that of an equivalent intravenous dose of the GABA_{B} agonist, greater than about 20%, greater than about 40% and in certain embodiments greater than about 60%.

Controlled release dosage forms comprising (3R)-4-{[(1S)-2-methyl-1-(2-methylpropanoyloxy)propoxy]carbonylamino}-3-(4-chlorophenyl)butanoic acid are disclosed by Leung et al., US 2008/0206332; and Sastry et al., U.S. application Ser. No. 12/024,830 filed Feb. 1, 2008, each of which is incorporated by reference herein in its entirety.

### Methods of Use

Prodrugs of GABA_{B} agonists that provide a high oral bioavailability of the corresponding GABA_{B} agonist and dosage forms comprising such prodrugs of GABA_{B} agonists may be used to treat neuropathic or musculoskeletal pain. Methods provided by the present disclosure comprise treating neuropathic or musculoskeletal pain in a patient by administering to a patient in need of such treatment a therapeutically effective dose of at least one prodrug of a GABA_{B} agonist that provides a high oral bioavailability of the corresponding GABA_{B} agonist. In certain embodiments, methods provided by the present disclosure do not comprise pain caused by spasticity, such as, for example, painful spasms associated with spasticity.

### Neuropathic Pain

It is estimated that neuropathic pain affects over 6 million patients in the U.S. and Europe and over 26 million patients worldwide. Neuropathic pain involves an abnormal processing of sensory input usually occurring after direct injury or damage to nerve tissue. Neuropathic pain is a collection of disorders characterized by different etiologies including infection, inflammation, disease such as diabetes and multiple sclerosis, trauma or compression to major peripheral nerves, and chemical or irradiation-induced nerve damage. Neuropathic pain typically persists long after tissue injury has resolved.

Prodrugs of GABA_{B} agonists provided by the present disclosure can be used to treat neuropathic pain. In certain embodiments, prodrugs of GABA_{B} agonists provided by the present disclosure can be used to treat neuropathic pain including, for example, post-herpetic neuralgia, peripheral neuropathy, trigeminal neuralgia, painful diabetic neuropathy, HIV-related neuropathic pain, cancer-related pain, or fibromyalgia.

International Association for the Study of Neuropathic Pain defines neuropathic pain states as disorders that are characterized by lesions or dysfunction of the neural system(s) that under normal conditions transmit noxious information to the central nervous system. The mechanisms underlying neuropathic pain conditions are highly heterogeneous, however, all types of neuropathic pain are presumed to involve nerve injury and certain common aberrations in somatosensory processing in the central and/or peripheral nervous system. Potential causes of neuropathic pain include physical damage, infection, and chemical exposure. Neuropathic pain can be generally classified as a focal/multifocal lesion of the peripheral nervous system, e.g., post-herpetic neuralgia, a generalized lesion of the peripheral nervous system, e.g., painful diabetic neuropathy, HIV-related NP), a lesion of the central nervous system, or a more complex neuropathic disorder. Peripheral neuropathic pain can arise as a consequence of trauma and surgery related nerve injury, e.g., brachial plexus injury; entrapment neuropathies such as lumbar disc compression, carpal tunnel syndrome; disease-related neuropathies, e.g., diabetes and HIV-AIDS; radiculopathy; complex regional pain syndrome; and/or tumor growth leading to nerve compression or infiltration. Central neuropathic pain can be the result of stroke, multiple sclerosis, post-ischemic myelopathy; post-herpetic neuralgia; and/or post-traumatic spinal cord injury.

Neuropathic pain can be characterized as a partial or complete loss of afferent sensory function and the paradoxical presence of certain hyperphenomena in the painful area. The nerve tissue lesion may be found in the brain, spinal cord, or the peripheral nervous system. Symptoms vary depending on the condition and can manifest as hyperalgesia (the lowering of pain threshold and an increased response to noxious stimuli), allodynia (the evocation of pain by non-noxious stimuli such as cold, warmth, or touch), hyperpathia (an explosive pain response that is suddenly evoked from cutaneous areas with increased sensory detection threshold when the stimulus intensity exceeds sensory threshold), paroxysms (a type of evoked pain characterized by shooting, electric, shock-like or stabbing pain that occur spontaneously, or following stimulation by an innocuous tactile stimulus or by a blunt pressure), paraesthesia (abnormal but non-painful sensations, which can be spontaneous or evoked, often described as pins and needles), dysesthesia (abnormal unpleasant but not necessarily painful sensation, which can be spontaneous or provoked by external stimuli), referred pain and abnormal pain radiation (abnormal spread of pain), and wind-up like pain and aftersensations (the persistence of pain long after termination of a painful stimulus).

Patients with neuropathic pain typically describe burning, lancinating, stabbing, cramping, aching, and sometimes vice-like pain. The pain can be paroxysmal or constant. Pathological changes to the peripheral nerve(s), spinal cord, and brain have been implicated in the induction and maintenance of chronic neuropathic pain. Patients suffering from neuropathic pain typically endure chronic, debilitating episodes that are refractory to current pharmacotherapies and profoundly affect their quality of life. Currently available treatments for neuropathic pain, including tricyclic antidepressants and gabapentin, typically show limited efficacy in the majority of patients.

There are several types of neuropathic pain. A classification that relates to the type of damage or related pathophysiology causing a painful neuropathy includes neuropathies associated with mechanical nerve injury such as carpal tunnel syndrome, vertebral disk herniation, entrapment neuropathies, ulnar neuropathy, and neurogenetic thoracic outlet syndrome; metabolic disease associated neuropathies such as diabetic polyneuropathy; neuropathies associated with neurotropic viral disease such as herpes zoster and human immunodeficiency virus (HIV) disease; neuropathies associated with neurotoxicity such as chemotherapy of cancer or tuberculosis, radiation therapy, drug-induced neuropathy, and alcoholic neuropathy; neuropathies associated with inflammatory and/or immunologic mechanisms such as multiple sclerosis, anti-sulfatide antibody neuropathies, neuropathy associated with monoclonal gammopathy, Sjogren's disease, lupus, vasculitic neuropathy, polyclonal inflammatory neuropathies, Guillain-Barre syndrome, chronic inflammatory demyelinating neuropathy, multifocal motor neuropathy, paraneoplastic autonomic neuropathy, ganglinoic acetylcholine receptor antibody autonomic neuropathy, Lambert-Eaton myasthenic syndrome and myasthenia gravis; neuropathies associated with nervous system focal ischemia such as thalamic syndrome (anesthesia dolorosa); neuropathies associated with multiple neurotransmitter system dysfunction such as complex regional pain syndrome (CRPS); neuropathies associated with chronic/neuropathic pain such as osteoarthritis, low back pain, fibromyalgia, cancer bone pain, chronic stump pain, phantom limb pain, and paraneoplastic neuropathies; toxic neuropathies (e.g., exposure to chemicals such as exposure to acrylamide, 3-chlorophene, carbamates, carbon disulfide, ethylene oxide, n-hexane, methyl n-butylketone, methyl bromide, organophosphates, polychlorinated biphenyls, pyriminil, trichlorethylene, or dichloroacetylene), focal traumatic neuropathies, phantom and stump pain, monoradiculopathy, and trigeminal neuralgia; and central neuropathies including ischemic cerebrovascular injury (stroke), multiple sclerosis, spinal cord injury, Parkinson's disease, amyotrophic lateral sclerosis, syringomyelia, neoplasms, arachnoiditis, and post-operative pain; mixed neuropathies such as diabetic neuropathies (including symmetric polyneuropathies such as sensory or sensorimotor polyneuropathy, selective small-fiber polyneuropathy, and autonomic neuropathy; focal and multifocal neuropathies such as cranial neuropathy, limb mononeuropathy, trunk mononeuropathy, mononeuropathy multiplex, and asymmetric lower limb motor neuropathy) and sympathetically maintained pain. Other neuropathies include focal neuropathy; glosopharyngeal neuralgia; ischemic pain; trigeminal neuralgia; atypical facial pain associated with Fabry's disease, Celiac disease, hereditary sensory neuropathy, or B₁₂-deficiency; mono-neuropathies; polyneuropathies; hereditary peripheral neuropathies such as Carcot-Marie-Tooth disease, Refsum's disease, Strumpell-Lorrain disease, and retinitis pigmentosa; acute polyradiculoneuropathy; and chronic polyradiculoneuropathy. Paraneoplastic neuropathies include paraneoplastic subacute sensory neuropathy, paraneoplastic motor neuron disease, paraneoplastic neuromyotonia, paraneoplastic demyelinating neuropathies, paraneoplastic vasculitic neuropathy, and paraneoplastic autonomic insufficiency. Prodrugs of GABA_{B} agonists provided by the present disclosure can be used to treat any of the foregoing types of neuropathic pain. In certain embodiments, the neuropathic pain is chosen from post-herpetic neuralgia, peripheral neuropathy, trigeminal neuralgia, painful diabetic neuropathy, HIV-related neuropathic pain, cancer-related pain, and fibromyalgia. In certain embodiments, the neuropathic pain is chosen from post-herpetic neuralgia and trigeminal neuralgia.

GABA is the major inhibitory neurotransmitter in the vertebrate central nervous system. GABA receptors have been classified into three distinct subtypes GABA_{A}, GABA_{B}, and GABA_{C}. Both the GABA_{A} and GABA_{C} receptors form ligand-gated chloride channels, while the GABA_{B} receptor belongs to the G-protein-coupled receptor family in which activation causes a decrease in Ca²⁺ and increase in K⁺ membrane conductance.

GABA_{B} receptors are found in the spinal cord, mainly in laminae I through III of the dorsal horn, and on presynaptic terminals of primary afferent neurons and inhibitory interneurons. GABA_{B} receptors are also located in the periphery. GABA_{B} agonists act by increasing potassium conductance through a G-protein mechanism and a second-messenger system, thereby producing membrane hyperpolarization. The action on the potassium channel occurs within the central nervous system and is mainly postsynaptic, producing hyperpolarization of second-order neurons. In addition, GABA_{B} receptor activation leads to inhibition of calcium conductance across voltage-gated Ca²⁺ channels. This effect has been demonstrated in dorsal root ganglion cells and, to some extent, explains the ability of GABA_{B} agonists such as baclofen to decrease the evoked release of excitatory neurotransmitters. Baclofen has been shown to inhibit the release of both glutamate and substance P from primary afferent nerve terminals.

Baclofen is a stereospecifically active agonist at the GABA_{B} receptor. It has been used as a muscle relaxant. A mechanism underlying the antinociceptive action of baclofen within the spinal cord appears to derive from suppression of the release of primary afferent transmitter. Activation of peripheral GABA_{B} receptors by baclofen induces antinociception via the opening of the voltage-dependent K⁺ channel or the G-protein-coupled inwardly rectifying K⁺ channel.

The GABA_{B} agonist (R,S)-baclofen has long been known to have antinociceptive activity in models of acute pain and recent studies have shown that baclofen inhibits allodynia and hyperalgesia in the chronic constriction injury and spinal nerve ligation models of persistent neuropathic pain at doses lower than those required to produce sedation and impairment of motor activity. However, GABA_{B} receptors are also located in the ventral horn of the spinal cord where they have an inhibitory effect on motor neurons resulting in muscle relaxation. Thus, in the absence of a clear analgesic therapeutic window, baclofen is primarily used clinically as a spasmolytic agent.

In clinical studies, intrathecal baclofen administration has been shown to be effective in treating neuropathic pain associated with spinal-cord injury and multiple sclerosis (Herman et al., Clin J Pain 1992, 12, 241-247; and Taira et al., Stereotactic Funct Neurosurg 1995, 65, 101-105), painful extremity paresthesias (Gatscher et al., Acta Neurochir Suppl 2002, 79, 75-76), sympathetically maintained pain (Van Hilten et al., N Engl J Med 2000, 343, 625-630; Becker et al., J Clin Neurosci 2000, 7, 316-319; and Zuniga et al., Reg Anesth Pain Med 2002, 27, 90-93). GABA_{B} agonists such as baclofen have also been shown to be effective in trigeminal, gloospharyngeal, vagoglossopharyngeal, and ophthalmic-postherpetic neuralgias (Bowsher, Br Med Bull 1991, 47, 655-66; Fromm et al., Neurology 1981, 31, 683-687; and Ringel and Roy, Ann Neurol 1987, 21, 514-515); and in patients with diabetic neuropathy (Anghinah et al., Muscle Nerve 1994, 958-59). Doses of baclofen from about 50 mg/day to about 60 mg/day have been shown to be effective in treating trigeminal neuralgia (Fromm et al., Ann Neurol 1984, 15, 240-244).

The efficacy of prodrugs of GABA_{B} agonists provided by the present disclosure for treating various types of neuropathic pain can also be assessed in clinical trials using, for example, using randomized double-blind placebo controlled methods. End points used in clinical trials for neuropathic pain can be determined using validated neuropathic pain criteria such as the Brief Pain Inventory, Categorical Scale, Gracety Pain Scale, Likert Scale, Neuropathic Pain Scale, Numerical Pain Scale, Short Form McGill Pain Questionnaire, Verbal Pain Scale, Visual Analog Scale (VAS), VAS Pain Intensity Scale, and/or VAS Pain Relief Scale.

### Musculoskeletal Pain

Musculoskeletal conditions causing tenderness and muscle spasms include fibromyalgia, tension headaches, myofascial pain syndrome, facet joint pain, internal disk disruption, somatic dysfunction, spinal fractures, vertebral osteomyelitis, polymyalgia rheumatica, atlantoaxial instability, atlanto-occipital joint pain, osteoporotic vertebral compression fracture, Scheuermann's disease, spondyloysis, spondylolisthesis, kissing spines, sacroiliac joint pain, sacral stress fracture, coccygodynia, failed back syndrome, and mechanical low back or neck pain (Meleger and Krivickas, Neurol Clin 2007, 25, 419-438. In these conditions, muscle spasm is related to local factors involving the affected muscle groups without the increased tone or reflex characteristic of spasticity. Muscle, tendon, ligament, intervertebral disc, articular cartilage, and bone can be involved in musculoskeletal pain. Disorders that can produce neck and back pain include muscle strain, ligament sprain, myofascial pain, fibromyalgia, facet joint pain, internal disc disruption, somatic dysfunction, spinal fracture, verterbral osteomyelitis, and polymyalgia rheumatica, atlantoaxial instability and atlanto-occipital joint pain.

GABA_{B} agonists are known to induce muscle-relaxant effects when administered systemically or centrally (Malcangio and Bowery, Trends Pharmacol Sci 1996, 17, 457-462). Consequently, the use of GABA_{B} agonists such as baclofen for treating spasticity associated with upper motor neuron syndromes is well established. Studies have also shown that GABA_{B} agonists can be effective in treating muscular pain and/or spasms associated with peripheral musculoskeletal conditions. Baclofen has been shown effective in treating migraine (Hering-Hanit, Cephalalgia 1999, 19, 589-591; and Hering-Hanit and Gadoth, Headache 2000, 40, 48-51); and specifically in tension-type headaches (Freitag, CNS Drugs 2003, 17(6), 373-381); as well as in low-back pain and radiculopathy (Zuniga et al., Anesthesiology 2000, 92, 876-880; Vatine et al., Pain Clin 1989, 2, 207-217; Dapas et al., Spine 1985, 10(4), 345-349; Raphael et al., BMC Musculoskeletal Disorders 2002, 3(17); and Magora et al., Pain Clin 1988, 2, 81-85).

The efficacy of prodrugs of GABA_{B} agonists provided by the present disclosure for treating one or more types of musculoskeletal pain can be assessed in animal models of neuropathic pain and in clinical trials. Kehl et al, disclose an animal model of muscle hyperplasia that employs intramuscular injection of carrageenan as useful for assessing the mechanisms and management of musculoskeletal pain (Kehl et al., Pain 2000, 85, 333-343).

### Back Pain

Prodrugs of GABA_{B} agonists provided by the present disclosure can be used to treat back pain including back pain in the cervical, thoracic, and/or lumbar spinal regions. The back pain may be acute or chronic. Acute low back pain is defined as low back pain present for fewer than 4 weeks, sometimes grouped with subacute low back pain as symptoms present for fewer than 3 months. Chronic low back pain is defined as low back pain present for more than 3 months.

### Low Back Pain

Low back pain generally occurs in the lumbar region of the back in the location of lumbar vertebrae L1-L5. Pain in the lower back can be caused by a sprain, strain, or spasm to one of the muscles, ligaments, facet joints, and/or sacroiliac joints in the back; spinal sprain or overcompression; or disc rupture or bulge. Low back pain may also reflect nerve or muscle irritation or bone lesions. Most low back pain follows injury or trauma to the back, but pain may also be caused by degenerative conditions such as arthritis or disc disease, osteoporosis, or other bone diseases, viral infections, irritation to joints and discs, or congenital abnormalities in the spine. Obesity, smoking, weight gain during pregnancy, stress, poor physical condition, posture inappropriate for the activity being performed, and poor sleeping position also may contribute to low back pain. Additionally, scar tissue created when the injured back heals itself does not have the strength or flexibility of normal tissue. Buildup of scar tissue from repeated injuries eventually weakens the back and can lead to more serious injury. Occasionally, low back pain may indicate a more serious medical problem. Pain accompanied by fever or loss of bowel or bladder control, pain when coughing, and progressive weakness in the legs may indicate a pinched nerve or other serious condition. People with diabetes may have severe back pain or pain radiating down the leg related to neuropathy. Low back pain can be caused by bulging disc (e.g., protruding, herniated, or ruptured disc), sciatica, spinal degeneration, spinal stenosis, osteoporosis, osteoarthritis, compression fractures, skeletal irregularities, fibromyalgia, spondylolysis and/or spondylolisthesis. Less common spinal conditions that can cause low back pain include ankylosing spondylitis, bacterial infections, osteomyelitis, spinal tumors, Paget's disease, and Scheuermann's disease. Clinical results suggest that GABA_{B} agonists such as baclofen can be effective in treating low back pain (Dapas et al., Spine 1985, 10(4), 345-349; and Raphael et al., BMC Musculoskeletal Disorders 2002, 3917). For example doses of baclofen from about 20 mg/day to about 80/mg day have been shown to be effective in treating acute low back pain (Dapas et al., Spine 1985, 10(4), 345-9).

In certain embodiments, methods of treating low back pain provided by the present disclosure comprises treating disorders, conditions, and/or symptoms associated with low back pain such as muscle spasms. Symptoms of low back pain can depend on the cause. For example, symptoms of back sprain or back strain include muscle spasms, cramping, stiffness, and pain centered in the back and buttocks. Symptoms of nerve-root pressure include leg pain, also referred to as sciatica, and nerve-related manifestations such as tingling, numbness, or weakness in one leg or in the foot, lower leg, or both legs. Symptoms of arthritis of the spine include pain and stiffness that are worse in the back and hip.

Muscle Spasm Associated with Acute Painful Musculoskeletal Conditions

Muscle spasms are associated with many acute painful musculoskeletal conditions. Low back pain and neck pain are common manifestations of such conditions. Acute musculoskeletal spasm of the back is a common disorder that causes localized pain, stiffness, reduced mobility, impaired activities of daily living, and sleep disturbances. Most episodes of acute low back pain or neck pain are nonspecific. Most subjects do not meet the criteria set forth for low back and neck pain, including significant trauma, cancer, infection, or motor weakness. Nonspecific back pain is defined as mechanical back pain, facet joint pain, osteoarthritis, muscle sprains, and muscle spasms. Low back pain may be caused by reflex spasms in the paraspinal muscles. Acute back spasms are involuntary, and often painful contractions of the muscles of the back including the cervical, thoracic, and/or lumbar spinal regions. Spasms associated with the lumbar vertebrae are also referred to as lower back spasms.

Typical pharmacologic treatments for acute neck and low back pain are NSAIDS, acetaminophen, and muscle relaxants. A recent placebo-controlled study concluded that baclofen was effective, safe, and well-tolerated in treating acute low-back syndrome with evidence of paravertebral muscle spasm and functional disability of less than 2 weeks duration (Dapas et al., Spine 1985, 10(4), 345-349). Accordingly prodrugs of GABA_{B} agonists provided by the present disclosure can be used to treat muscle spasm associated with acute painful musculoskeletal conditions, including acute back spasms, and more particularly acute lower back spasms.

### Fibromyalgia

Fibromyalgia is a condition characterized by aching and pain in muscles, tendons and joints all over the body, but especially along the spine. The body also is tender to touch in specific areas referred to as tender or trigger points. Other symptoms of fibromyalgia include sleep disturbance, depression, daytime tiredness, headaches, alternating diarrhea and constipation, numbness and tingling in the hands and feet, feelings of weakness, memory difficulties, and dizziness. Although the etiology of fibromyalgia is not known, stress, disordered sleep patterns, abnormal production of pain-related chemicals in the nervous system, and/or low levels of growth hormone are believed to contribute to the onset of fibromyalgia.

Current treatment of fibromyalgia is based on symptoms, with the goal of alleviating pain, restoring sleep, and improving general quality of life. Several nonpharmacologic treatments include exercise, education, behavioral and physical therapy. Pharmacologic treatments include tricyclic compounds, serotonin reuptake inhibitors, analgesics, muscle relaxants, and ACE inhibitors. There is evidence suggesting that GABA_{B} agonists such as baclofen may be useful in improving fibromyalgia symptoms (Taylor-Gjevre and Gjevre, Lupis 2005, 14(6), 486-8).

The efficacy of administering compounds provided by the present disclosure for treating fibromyalgia may be assessed using animal and human models of fibromyalgia and in clinical trials. Animal models of neuropathic pain or clinically relevant studies of different types of neuropathic pain have been found useful in assessing therapeutic activity for treating fibromyalgia.

### Dose

The amount of a prodrug of a GABA_{B} agonist that will be effective in treating neuropathic or musculoskeletal pain will depend on the nature of the disease, disorder, or condition, and can be determined by standard clinical techniques known in the art. In addition, in vitro or in vivo assays may optionally be employed to help identify optimal dosage ranges. The amount of a compound administered can depend on, among other factors, the patient being treated, the weight of the patient, the health of the patient, the disease being treated, the severity of the affliction, the route of administration, the potency of the compound, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

In certain embodiments, a therapeutically effective dose of a prodrug of a GABA_{B} agonist for treating neuropathic or musculoskeletal pain may comprise about 1 mg-equivalents to about 2,000 mg-equivalents of the corresponding GABA_{B} agonist per day, about 5 mg-equivalents to about 1000 mg-equivalents of the corresponding GABA_{B} agonist per day, about 10 mg-equivalents to about 500 mg-equivalents of the corresponding GABA_{B} agonist per day, and in certain embodiments, about 10 mg-equivalents to about 100 mg-equivalents of the corresponding GABA_{B} agonist per day.

A dose may be administered in a single dosage form or in multiple dosage forms. When multiple dosage forms are used the amount of a prodrug of a GABA_{B} agonist contained within each of the multiple dosage forms may be the same or different.

In certain embodiments, an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. A dose of a highly orally bioavailable prodrug of a GABA_{B} agonist may be within a range of circulating concentrations in for example the blood, plasma, or central nervous system, that is therapeutically effective, that is less than a sedative dose, and that exhibits little or no toxicity. A dose may vary within this range depending upon the dosage form employed.

During treatment a dose and dosing schedule may provide sufficient or steady state systemic concentrations of a therapeutically effective amount of a GABA_{B} agonist to treat a disease. In certain embodiments, an escalating dose may be administered. Prodrugs of GABA_{B} agonists that provide a high oral bioavailability of the corresponding GABA_{B} agonist may be administered orally, and may be administered at intervals for as long as necessary to obtain an intended or desired therapeutic effect.

### Combination Therapy

Prodrugs of GABA_{B} agonists that provide a high oral bioavailability of the corresponding GABA_{B} agonist may be used in combination therapy with at least one other therapeutic agent. In certain embodiments, prodrugs of GABA_{B} agonists provided by the present disclosure and pharmaceutical compositions thereof may be administered to a patient for treating neuropathic pain in combination with a therapy or another therapeutic agent known or believed to be effective in treating neuropathic pain. Prodrugs of GABA_{B} agonists and another therapeutic agent(s) can act additively or, and in certain embodiments, synergistically. In some embodiments, prodrugs of GABA_{B} agonists may be administered concurrently with the administration of another therapeutic agent, such as for example, a compound for treating neuropathic or musculoskeletal pain. In some embodiments, prodrug of GABA_{B} agonists may be administered prior or subsequent to administration of another therapeutic agent, such as for example, a compound for treating neuropathic or musculoskeletal pain.

Methods provided by the present disclosure include administering one or more prodrugs of GABA_{B} agonists and one or more other therapeutic agents provided that the combined administration does not inhibit the therapeutic efficacy of the one or more prodrugs of GABA_{B} agonists and/or other therapeutic agent and/or does not produce adverse combination effects.

In certain embodiments, prodrugs of GABA_{B} agonists may be administered concurrently with the administration of another therapeutic agent, which may be part of the same pharmaceutical composition or dosage form as or in a different composition or dosage form than that containing a prodrug of a GABA_{B} agonist. When a prodrug of a GABA_{B} agonist is administered concurrently with another therapeutic agent that potentially can produce adverse side effects including, but not limited to, toxicity, the therapeutic agent may be administered at a dose that falls below the threshold at which the adverse side effect is elicited.

In certain embodiments, prodrugs of GABA_{B} agonists may be administered prior or subsequent to administration of another therapeutic agent. In certain embodiments of combination therapy, the combination therapy comprises alternating between administering a prodrug of a GABA_{B} agonist and a composition comprising another therapeutic agent, e.g., to minimize adverse side effects associated with a particular drug.

Examples of drugs useful for treating pain include opioid analgesics such as morphine, codeine, fentanyl, meperidine, methadone, propoxyphene, levorphanol, hydromorphone, oxycodone, oxymorphone, tramadol and pentazocine; nonopioid analgesics such as aspirin, ibuprofen, ketoprofen, naproxen, and acetaminophen; non-steroidal anti-inflammatory drugs such as aspirin, choline magnesium trisalicylate, diflunisal, salsalate, celecoxib, rofecoxib, valdecoxib, diclofenac, etodolac, fenoprofen, flubiprofen, ibuprofen, indomethacin, ketoprofen, ketorolac, meclofanamate, mefenamic acid, meloxicam, nabumetone, naproxen, oxaprozin, piroxicam, sulindac, and tometin; antiepileptics such as gabapentin, pregabalin, carbamazepine, phenyloin, lamotrigine, and topiramate; antidepressants such as duloxetine, amitriptyline, venlafaxine, nortryptyline, imipramine, and desipramine; local anesthetics such as lidocaine, and mexiletine; NMDA receptor antagonists such as dextropethorphan, memantine, and ketamine; N-type calcium-channel blockers such as ziconotide; vanilloid receptor-1 modulators such as capsaicin; cannabinoid receptor modulators such as sativex; neurokinin receptor antagonists such as lanepitant; other analgesics such as neurotropin; and other drugs such as desipramine, clonazepam, divalproex, oxcarbazepine, divalproex, butorphanol, valdecoxib, vicoprofen, pentazocine, propoxyhene, fenoprofen, piroxicam, indometnacin, hydroxyzine, buprenorphine, benzocaine, clonidine, flurbiprofen, meperidine, lacosamide, desvenlafaxine, and bicifadine.

In certain embodiments, a drug useful for treating neuropathic pain is chosen from propoxyphene, meperidine, hydromorphone, hydrocodone, morphine, codeine, 2-piperidinol-1-alkanol, eliprodil, ifenprodil, rofecoxib, celecoxib, salicylic acid, diclofenac, piroxicam indomethacin, ibuprofen, naproxen, gabapentin, carbemazepine, pregabalin, topiramate, valproic acid, sumatriptan, elitriptan, rizatriptan, zolmitriptan, naratriptan, flexeril, carisoprodol, robaxisal, norgesic, dantrium, diazepam, chlordiazepoxide, alprazolam, lorazepam, acetaminophen, nitrous oxide, halothane, lidocaine, etidocaine, ropivacaine, chloroprocaine, sarapin, bupivacaine, capsicin, desipramine, amitriptyline, doxepin, perphenazine, protriptyline, tranylcypromine, baclofen, clonidine, mexelitine, diphenhydramine, hydroxyzine, caffeine, prednisone, methyl-prednisone, decadron, sertraline, paroxetine, fluoxetine, tramadol, levodopa, dextromethorphan, substance P antagonists, and botulinum toxin.

Non-pharmacological therapies for treating neuropathic pain include transcutaneous electrical nerve stimulation, percutaneous electrical nerve stimulation, and acupuncture.

In certain embodiments, prodrugs of GABA_{B} agonists provided by the present disclosure and pharmaceutical compositions thereof may be administered to a patient for treating fibromyalgia in combination with a therapy or another therapeutic agent known or believed to be effective in treating fibromyalgia, or in certain embodiments, a disease, disorder, or condition associated with fibromyalgia. Drug therapy for fibromyalgia may be tailored to the severity and frequency of fibromyalgia episodes. For occasional episodes, acute treatment may be indicated. For fibromyalgia episodes occurring two or more times per month, or when attacks greatly impact the patient's daily life, chronic therapy on an ongoing basis may be appropriate.

Treatments for fibromyalgia that reduce the frequency of episodes and include non-steroidal anti-inflammatory agents (NSAIDs), adrenergic beta-blockers, calcium channel blockers, tricyclic antidepressants, selective serotonin reuptake inhibitors, anticonvulsants, NMDA receptor antagonists, dopamine agonists, selective 5-HT₃ receptor antagonists, opioids, muscle relaxants, sedative hypnotics, and other therapy. Examples of NSAIDs useful for treating fibromyalgia include aspirin, ibuprofen, fenoprofen, flurbiprofen, ketoprofen, mefenamic acid, and naproxen. Examples of adrenergic beta-blockers useful for treating fibromyalgia include acebutolol, atenolol, imilol, metoprolol, nadolol, pindolol, propranolol, and timolol. Examples of calcium channel blockers useful for treating fibromyalgia include amlodipine, diltiazem, dotarizine, felodipine, flunarizine, nicardipine, nifedipine, nimodipine, nisoldipine, and verapamil. Examples of tricyclic antidepressants useful for treating fibromyalgia include amitriptyline, desipramine, doxepin, imipramine, nortriptyline, cyclobenzaprine, and protriptyline. Examples of selective serotonin reuptake inhibitors useful for treating fibromyalgia include fluoxetine, methysergide, nefazodone, paroxetine, sertraline, citalopram, and venlafaxine. Examples of other antidepressants useful for treating g fibromyalgia include bupropion, nefazodone, norepinephrine, venlafaxine, duloxetine, and trazodone. Examples of anticonvulsants (antiepileptics) useful for treating fibromyalgia include divalproex sodium, felbamate, gabapentin, lamotrigine, levetiracetam, oxcarbazepine, tiagabine, topiramate, valproate, and zonisamide. Examples of NMDA receptor antagonists useful for treating fibromyalgia include dextromethorphan, magnesium, and ketamine. Examples of dopamine agonists useful for treating fibromyalgia include α-dihydroergocryptine. Examples of opioids useful for preventing fibromyalgia are tramadol, oxycodone, and methadone. An example of a muscle relaxant useful for treating fibromyalgia is cyclobenzaprine. Examples of therapies useful for treating fibromyalgia include exercise, interferon, growth hormone, hormone therapy, diet low in animal fat and high in fiber, and complementary therapies such as counseling/psychotherapy, relaxation training, progressive muscle relaxation, guided imagery, diaphragmatic breathing, biofeedback, acupuncture, and physical and massage therapy.

Acute fibromyalgia treatments intended to eliminate or reduce the severity of muscular/skeletal pain and any associated symptoms include serotonin receptor agonists, such as triptans (5-hydroxytryptophan (5-HT) agonists), for example, almotriptan, eletriptan, frovatriptan, naratriptan, rizatriptan, sumatriptan, and zolmitriptan; ergotamine-based compounds such as dihydroergotamine and ergotamine; antiemetics such as metoclopramide and prochlorperazine; and compounds that provide analgesic effects.

Other examples of drugs useful in treating fibromyalgia include acetaminophen-aspirin, caffeine, cyproheptadine, methysergide, valproic acid, NSAIDs such as diclofenac, flurbiprofen, ketaprofen, ketorolac, ibuprofen, indomethacin, meclofenamate, and naproxen sodium, opioids such as codeine, meperidine, and oxycodone, and glucocorticoids such as dexamethasone, prednisone, and methylprednisolone.

Prodrugs of GABA_{B} agonists provided by the present disclosure can also be administered in conjunction with drugs that are useful for treating symptoms associated with fibromyalgia such as migraine headache, and depression. Examples of therapeutic agents useful for treating migraine include beta-blockers such as atenolol, metoprolol, proranolol, timolol, and nadolol; NSAIDS such as fenoprofen, flurbiprofen, ketoprofen, and naproxen; calcium channel blockers such as verapamil, diltiazem, nicardipine, nifedipine, and nimodipine; anti-epilepsy medication such gabapentin, divalproex sodium, and topiramate; tricyclic antidepressants such as amitriptyline, doxepin, imipramine, nortriptlyine, protriptyline, and desipramine; serotonin reuptake inhibitors such as fluoxetine, sertraline, paroxetine, nefazodone, and venlafazine. Examples of therapeutic agents useful for treating depression include tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as fluoxetine, fluvoxamine, paroxetine, and setraline; monoamine oxidase inhibitors such as isocarboxazid, pargyline, phenizine, and tranylcypromine; and psychostimulants such as dextroamphetamine and methylphenidate.

In certain embodiments, prodrugs of GABA_{B} agonists provided by the present disclosure and pharmaceutical compositions thereof may be administered to a patient for treating musculoskeletal pain in combination with a therapy or another therapeutic agent known or believed to be effective in treating musculoskeletal pain.

Examples of drugs useful for treating musculoskeletal pain include cyclobenzaprine, dantrolene, methocarbamol, orphenadrine, tizanidrine, metaxalone, carisoprodol, chlorphenesin, chlorzoxazone, alprazolam, bromazepam, chlordiazepoxide, clorazepate, diazepam, flunitriazepam, lorazepam, medazepam, midazolam, oxazepam, prazepam, triazolam, temazepam, tolperisone, thiocolchicoside, tetrazepam, afloqualone, pridinol, tapentadol, and botulinum toxin. In certain embodiments, any of the drugs useful for treating neuropathic pain may be coadministered with a prodrug of a GABA_{B} agonist for treating musculoskeletal pain.

In certain embodiments, prodrugs of GABA_{B} agonists provided by the present disclosure and pharmaceutical compositions thereof may be administered to a patient for treating low back pain in combination with a therapy or another therapeutic agent known or believed to be effective in treating low back pain.

Examples of drugs useful for treating low back pain include NSAIDs such as aspirin, naproxen, and ibuprofen; anticonvulsants, antidepressants such as amitriptyline and desipramine; and opioids such as codeine, oxycodone, hydrocodone, and morphine. In certain embodiments, any of the drugs useful for treating neuropathic pain may be coadministered with a prodrug of a GABA_{B} agonist for treating low back pain.

Therapies for low back pain include the use of cold and hot compresses, bed rest, exercise, spinal manipulation, acupuncture, biofeedback, interventional therapy, traction, transcutaneous electrical nerve stimulation, ultrasound, vertebroplasty, kyphoplasty, discectomy, foraminotomy, intradiscal electrothermal therapy, nucleoplasty, radiofrequency lesioning, spinal fusion, and spinal laminectomy.

In certain embodiments, prodrugs of GABA_{B} agonists provided by the present disclosure and pharmaceutical compositions thereof may be administered to a patient for treating low back pain in combination with a therapy or other therapeutic agent for treating muscle spasms, for example muscle spasms associated with low back pain, such as muscle relaxants. Examples of drugs useful as muscle relaxants for treating muscle spasms include baclofen, carisoprodol, chlorzoxazone, cyclobenzaprine, diazepam, metaxalone, methocarbamol, orphenadrine, pentafluoropropane, eperisone, tolperisone, thiocolchicoside, tetrazepam, afloqualone, pridinol, chlorphenesin, tapentadol, and tizanidine.

In certain embodiments, prodrugs of GABA_{B} agonists provided by the present disclosure and pharmaceutical compositions thereof may be administered to a patient for treating neuropathic or musculoskeletal pain in combination with a colonically absorbable prodrug of a GABA analog, such as a colonically absorbable prodrug of gabapentin or pregabalin.

Colonically absorbable GABA analog prodrugs are disclosed in Gallop et al., U.S. Pat. No. 6,818,787, U.S. Pat. No. 6,972,341, U.S. Pat. No. 7,026,351, U.S. Pat. No. 7,060,727, U.S. Pat. No. 7,227,028, and US 2006/0122125; and Estrada et al., US 2005/0154057.

In certain embodiments, a colonically absorbable prodrug of gabapentin is chosen from a compound of Formula (V):

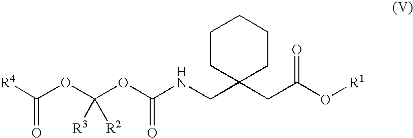

And pharmaceutically acceptable salts thereof, wherein:

R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a ring chosen from a cycloalkyl, substituted cycloalkyl, cycloheteroalkyl, and substituted cycloheteroalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In certain embodiments, a colonically absorbable prodrug of pregabalin is chosen from a compound of Formula (VI):

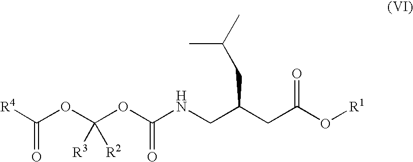

And pharmaceutically acceptable salts thereof, wherein:

R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a ring chosen from a cycloalkyl, substituted cycloalkyl, cycloheteroalkyl, and substituted cycloheteroalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In certain embodiments of compounds of Formula (V) and Formula (VI), each substituent is independently chosen from halogen, —OH, —CN, —CF₃, —C(O)NH₂, —COOR¹⁰, and —NR¹⁰₂ wherein each R¹⁰ is independently chosen from hydrogen and C₁₋₃ alkyl.

In certain embodiments of compounds of Formula (V) and Formula (VI), R¹ is hydrogen.

In certain embodiments of compounds of Formula (V) and Formula (VI), R² and R³ are independently chosen from hydrogen and C₁₋₆ alkyl.

In certain embodiments of compounds of Formula (V) and Formula (VI), one of R² and R³ is other than hydrogen.

In certain embodiments of compounds of Formula (V) and Formula (VI), R³ is chosen from methyl, ethyl, n-propyl, isopropyl, n-butyl, isobutyl, and sec-butyl; and R² is hydrogen.

In certain embodiments of compounds of Formula (V) and Formula (VI), R³ is chosen from methyl, ethyl, n-propyl, and isopropyl.

In certain embodiments of compounds of Formula (V) and Formula (VI), R⁴ is chosen from C₁₋₆ alkyl, and C₁₋₆ substituted alkyl. In certain embodiments of compounds of Formula (V) and Formula (VI) wherein R⁴ is chosen from C₁₋₆ substituted alkyl, the substituent group is chosen from halogen, —NH₂, —OH, —CN, —CF₃, —COOH, —C(O)NH₂, —C(O)OR¹⁰, and —NR¹⁰₂ wherein each R¹⁰ is independently C₁₋₃ alkyl.

In certain embodiments of compounds of Formula (V) and Formula (VI), R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formula (V) and Formula (VI), R⁴ is chosen from methyl, ethyl, n-propyl, isopropyl, n-butyl, and isobutyl.

In certain embodiments of compounds of Formula (V) and Formula (VI), each of R¹ and R² is hydrogen; R³ is C₁₋₆ alkyl; and R⁴ is chosen from C₁₋₆ alkyl and substituted C₁₋₆ alkyl. In certain embodiments of compounds of Formula (V) and Formula (VI), wherein each of R¹ and R² is hydrogen; R³ is C₁₋₆ alkyl; and R⁴ is chosen from C₁₋₆ alkyl and substituted C₁₋₆ alkyl, each substituent group is independently chosen from halogen, —NH₂, —OH, —CN, —CF₃, —COOH, —C(O)NH₂, —C(O)OR¹⁰, and —NR¹⁰₂ wherein each R¹⁰ is independently C₁₋₃ alkyl.

In certain embodiments of compounds of Formula (V) and Formula (VI), each of R¹ and R² is hydrogen; R³ is chosen from methyl, ethyl, n-propyl, isopropyl, n-butyl, isobutyl, and sec-butyl; and R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formula (V) and Formula (VI), each of R¹ and R² is hydrogen; R³ is chosen from methyl, ethyl, n-propyl, and isopropyl; and R⁴ is chosen from methyl, ethyl, n-propyl, isopropyl, n-butyl, and isobutyl.

In certain embodiments of the compound of Formula (V) wherein R⁴ is isopropyl, R² is hydrogen, and R³ is methyl; the compound of Formula (V) is 1-{[α-isobutanoyloxyethoxy)carbonyl]aminomethyl}-1-cyclohexane acetic acid, or pharmaceutically acceptable salt thereof.

In certain embodiments wherein R⁴ is isopropyl, R² is hydrogen, and R³ is methyl, a compound of Formula (VI) is 3-{[α-isobutanoyloxyethoxy)carbonyl]aminomethyl}-5-methyl hexanoic acid, or a pharmaceutically acceptable salt thereof.

In certain embodiments, a compound provided by the present disclosure may be administered to a patient together with a (3S)-aminomethyl-5-methyl-hexanoic acid prodrug as described by Yao and Gallop, U.S. Provisional Application Ser. Nos. 61/023,808 and 61/023,813, both filed Jan. 25, 2008, and each of which is incorporated by reference in its entirety.

In certain embodiments, a compound provided by the present disclosure may be administered to a patient together with the compound crystalline calcium (3S)-{[(1R)-isobutanoyloxyethoxy]carbonylaminomethyl}-5-methyl-hexanoate hydrate, which exhibits characteristic scattering angles (2θ) at least at 5.0°±0.2°, 7.4°±0.2°, 7.9°±0.2°, 11.6°±0.2°, 15.5°±0.2°, 17.2°±0.2°, and 19.0°±0.2° in an X-ray powder diffractogram measured using Cu—K_{α} radiation. In certain embodiments, the compound crystalline calcium (3S)-{[(1R)-isobutanoyloxyethoxy]carbonylaminomethyl}-5-methyl-hexanoate hydrate exhibits characteristic scattering angles (2θ) at least at 5.0°±0.2°, 7.4°±0.2°, 7.9°±0.2°, 11.6°±0.2°, 15.5°±0.2°, 16.3°±0.2°, 16.6°±0.2°, 17.2°±0.2°, 19.0°±0.2°, 22.2°±0.2°, and 24.9°±0.2° in an X-ray powder diffractogram measured using Cu—K_{α} radiation. In certain embodiments, the compound crystalline calcium (3S)-{[(1R)-isobutanoyloxyethoxy]carbonylaminomethyl}-5-methyl-hexanoate hydrate exhibits characteristic scattering angles (2θ) at least at 5.0°±0.2°, 7.0°±0.2°, 7.4°±0.2°, 7.9°±0.2°, 11.1°±0.2°, 11.6°±0.2°, 12.8°±0.2°, 13.7°±0.2°, 14.1°±0.2°, 15.1°±0.2°, 15.5°±0.2°, 16.3°±0.2°, 16.6°±0.2°, 17.2°±0.2°, 17.5°±0.2°, 17.8°±0.2°, 18.1°±0.2°, 18.4°±0.2°, 18.6°±0.2°, 19.0°±0.2°, 19.6°±0.2°, 19.8°±0.2°, 20.7°±0.2°, 21.0°±0.2°, 22.2°±0.2°, 23.1°±0.2°, 24.9°±0.2°, 26.1°±0.2°, 26.8°±0.2°, 27.8°±0.2°, 28.0°±0.2°, 28.8°±0.2°, 29.7°±0.2° and 30.5°±0.2° in an X-ray powder diffractogram measured using Cu—K_{α} radiation. In certain embodiments, the compound crystalline calcium (3S)-{[(1R)-isobutanoyloxyethoxy]carbonylaminomethyl}-5-methyl-hexanoate hydrate from comprises about 1 mole water per mole of the compound to about 3 moles water to mole of the compound. In certain embodiments, the compound crystalline calcium (3S)-{[(1R)-isobutanoyloxyethoxy]carbonylaminomethyl}-5-methyl-hexanoate hydrate from comprises from about 2 wt % water to about 5 wt % water.

In certain embodiments, a compound provided by the present disclosure may be administered to a patient together with calcium (3S)-{[(1R)-isobutanoyloxyethoxy]carbonylaminomethyl}-5-methyl-hexanoate, which exhibits characteristic scattering angles (2θ) at least at 5.4°±0.2° and 14.1°±0.2° in an X-ray powder diffractogram measured using Cu—K_{α} radiation, and in certain embodiments, which exhibits a melting point range from about 102° C. to about 111° C.

Methods of synthesizing colonically absorbable prodrugs of GABA analogs, including methods of synthesizing compounds of Formula (V) and (VI) are disclosed in Gallop et al., U.S. Pat. No. 6,818,787, U.S. Pat. No. 6,972,341, U.S. Pat. No. 7,186,855, U.S. Pat. No. 6,927,036; and Raillard et al., U.S. Pat. No. 7,232,924, U.S. Provisional Application Ser. Nos. 61/087,056 and 61/087,038, both filed Aug. 7, 2008; and Yao and Gallop, U.S. Provisional Application Ser. Nos. 61/023,808 and 61/023,813, both filed Jan. 25, 2008, each of which is incorporated by reference in its entirety.

### EXAMPLES

The following examples describe in detail methods of using prodrugs of GABA_{B} agonists provided by the present disclosure. It will be apparent to those skilled in the art that many modifications, both to materials and methods, may be practiced without departing from the scope of the disclosure.

### Example 1

### cAMP Assay for Determining GABA_{B} Receptor Agonist Activity

As an example of a cAMP assay for determining GABA_{B} receptor agonist activity the following procedure can be used. Recombinant HEK cells expressing the GABA_{B} R1a2 receptor are used. Cells are seeded overnight at 5,000 cells per well, in black, clear bottom 96 well plates. The following morning, cells are washed twice with 100 μL PBS per well. Forskolin is weighed and dissolved in dimethylsulfoxide (DMSO) to a final concentration 100 mM. One-hundred μM forskolin solutions are prepared in phosphate buffered saline (PBS) with and without test compound at 1-times final concentration. Thirty (30) μL of the test solutions are added to the wells and incubated for 1 h at room temperature. The cAMP concentration is detected according to the protocol described in a cAMP assay kit (for example, cAMP XS⁺ HitHunter™ Chemiluminescence Assay Kit (90-0075-02, GE Healthcare Biosciences Corp.), maintaining the plate at room temperature and in the dark. Two hours after the final kit reagent is added, the plate bottom is covered with black tape, and the plate read using a scintillation and luminescence counter (for example, 1450 MicroBeta Trilux microplate, PerkinElmer, Waltham, Mass.). Each well is read for 6 seconds and the data analyzed. A GABA_{B} receptor agonist produces a response by itself and the response is inhibited by a specific GABA_{B} receptor inhibitor such as (S-(R,R)-3-(((1-(3,4-dichlorophenyl)amino)-2-hydroxypropyl)(cyclohexylmethyl)phosphinic acid, hydrochloride (CGP54626). Furthermore, a GABA_{B} receptor agonist produces no response in oocytes that do not express the GABA_{B} receptor.

### Example 2

### Ca²⁺ Assay for Determining GABA_{B} Receptor Agonist Activity

As an example of a Ca²⁺ assay for determining GABA_{B} receptor agonist activity, the following procedure can be used to determine the GABA_{B} receptor agonist activity of a compound as reflected by activation of Ca²⁺ signaling. Human embryonic kidney cells expressing GABA_{B} R1a2 under tetracycline induction control (HEK TREx), and Gqi chimeric protein (expressed constitutively), allowing GABA_{B} R coupling through the Ca²⁺ signaling pathway are used. Cells are seeded in media containing tetracycline containing overnight at 100,000 cells/well, in black, clear-bottom, 96 well plates. The following morning, cells are washed twice with 100 μL Hank's balanced salt solution (HBSS) buffer per well. Fluorescent Ca⁺ indicator dye is prepared using the materials and procedure described in the F362056 Fluo-4 NW Calcium Assay Kit (Invitrogen, Carlsbad, Calif.). Ten (10) mL of kit buffer and 100 μl of kit Probenecid are added to individual kit dye vials, and rolled back and forth several times to dissolve the dye. Cells are then loaded into the dye solution at 50 μL per well. The cells and dye are incubated for 30 min at 37° C., and then incubated for an additional 30 min at room temperature in the dark. Test compounds are dissolved in HBSS buffer at twice final concentration. Duplicate wells are used for each unique condition. Solution containing the test compound is added to the wells. The fluorescence in each well is measured using an excitation wavelength 494 nm and a detection wavelength of 516 nm every 2 sec over a total time of 50 sec (for example, using a FLEXStation II (Molecular Devices, Sunnyvale, Calif.). A normalized fluorescence value for each well is calculated using the following procedure. The difference in fluorescence at 35 sec (usually representing maximal response) and at 15 sec (a time point prior to addition of test compounds) is calculated, divided by the fluorescence at 15 sec, and the result multiplied by 100. The final value represents the percent increase in fluorescence relative to the fluorescence at 15 sec. Data is analyzed using standard procedures. A GABA_{B} receptor agonist produces a response by itself and the response is inhibited by a specific GABA_{B} receptor inhibitor such as CGP54626. Furthermore, a GABA_{B} receptor agonist produces no response in oocytes that do not express the GABA_{B} receptor.

### Example 3

### Electrophysiology Assay for Determining GABA_{B} Receptor Agonist Activity

GABA_{B} receptor agonist activity can be determined using an electrophysiology method employing inward rectification of G-protein-coupled K⁺ channels (GIRK1/4) in Xenopus laevis oocytes expressing the GABA_{B} receptor (GABA_{B}R 1a/2). Expression of GABA_{B}R/GIRK in Xenopus laevis oocytes can be accomplished using the following procedure. Oocytes are removed from mature, anesthetized, HCG-injected female Xenopus laevis and washed in 0 mM CaCl₂ ND96 buffer (90 mM NaCl, 10 mM hemi-Na HEPES, 2 mM KCl, 1 mM MgCl₂). Oocytes are then shaken in collagenase solution for 1 h at room temperature. The oocytes are then washed thoroughly and sorted according to desired maturity and morphology. Selected oocytes are injected with a mixture of cRNA encoding for hGBBR1a+2 and rGIRK1+4. Final volume ratios of the GIRK1/4 and GBBR1a/2 RNA are about 1:10 and about 1:5, respectively. Forty-six (46) nL of the RNA mixture is injected into each oocyte. Uninjected oocytes are used as controls. Oocytes are incubated at 16-18° C. in 0.9 mM CaCl₂ ND96 buffer pH 7.4 (90 mM NaCl₂, 10 mM hemi-Na HEPES, 2 mM KCl, 1 mM MgCl₂, 0.9 mM CaCl₂) containing Pen/Strep (SV30010, Hyclone) for 1-2 days. Electrophysiology measurements are made using a 2-electrode voltage clamp recording instrument (for example, GeneClamp 500B amplifier/Clampex8.2/Clampfit8, Axon Instruments, Union City, Calif.) and analysis software (for example, Chart4, ADInstruments, Mountain View, Calif.). Dose response curves of GABA_{B} agonist activity and pEC50 values for a compound are determined as follows. Test compounds are weighed and dissolved in an appropriate solvent. Serial dilution curves are made in 100 mM KCl ND96 buffer (90 mM NaCl₂, 10 mM hemi-Na HEPES, 1 mM MgCl₂, 1.8 mM CaCl₂, 100 mM KCl). The highest concentration of a test compound is typically 1 mM, with 1:5 or 1:4 serial dilutions to provide a 5- or 6-point curve over a concentration range to 0.01 μM. Currents are measured with oocytes clamped at a holding potential between −15 mV to 40 mV, depending on the health and/or the receptor expression level of individual oocytes. Baseline currents at this holding potential are allowed to reach a steady state before addition of the test compound and recording.

Prior to and between each series of test compound dilutions, a sub-maximal concentration of a known GABA_{B} agonist (e.g., 4 μM GABA) is used as a control. Currents are measured by manually adding 650 μL of diluted test compound to a clamped oocyte in the holding chamber. Currents are allowed to saturate before activating the system vacuum/bath perfusion to wash away the test compound. If a test compound appears to have agonist activity, it is also tested in the presence of a known GABA_{B}R inhibitor 3-N[1-(S)-3,4-dichlorophenyl)ethylamino-2-(S)-hydroxypropyl-P-benzyl-phosphinic acid (CGP55845). Serial dilutions of the test compound are made in 100 mM KCl ND96 buffer containing 10 μM CGP55845. As another control, the test compound is also tested in uninjected oocytes at a single concentration of 100 μM. For analysis of the dose response curves, currents generated from each test dilution are calculated as a percentage of the current generated by the control compound. The curve traces are then analyzed and pEC50 values generated. A GABA_{B} receptor agonist produces a response by itself and the response is inhibited by a specific GABA_{B} receptor inhibitor such as CGP54626. Furthermore, a GABA_{B} receptor agonist produces no response in oocytes that do not express the GABA_{B} receptor.

### Example 4

### Hypothermia Model of GABA_{B} Agonist Activity

GABA_{B} receptor agonist activity of a compound can also be determined using animal models such as the hypothermia method described, for example by Queva et al., Br. J. Pharmacology 2003, 140, 315-322. In this method age-matched, C57B16/129Sv F1 hybrid GABA_{B(1)}^{+/+}, GABA_{B(1)}^{+/-} and GABA_{B(1)}^{-/-} mice are used. The mice are maintained incages at an ambient temperature between 21° C. and 23° C. and a relative humidity between 52% and 56%. A thermosensitive chip is implanted in the interscapular region under brief isoflurane anesthesia, and the animals are allowed to recover for at least 1 day. The animals have free access to food and water, except during the experiment. On the experimental day, the mice are place in individual cages at an ambient temperature of, for example about 20.5±1.0° C. After 30 min, three basal temperature recordings are made using a transponder communicating with a computer for data acquisition. In preliminary experiments, the system is evaluated in mice by measuring the interscapular temperature and rectal temperature at the same time. The thermosensitive chips are calibrated in the range from 32° C. to 43° C. against a thermistor in a water bath before implantation. The resolution of the chips is 0.1° C. Test compound or control are injected subcutaneously at an appropriate dose after the last measurement. The doses are chosen based on pilot experiments in which the doses are found to produce a significant hypothermia. Meaurements are then made at regular intervals. Behavioral scoring is made at each time point, and the behavioral data is presented as the maximal effect. The following definitions are used for behavioral effects: (1) no effect; (2) exophthalmus, slight motor impairment; (2) more pronounced motor impairment; (3) immobile with intact righting reflex; (4) no righting reflex, disturbed respiration, occasional seizures, detectable but very low muscle tonus; and (5) paralysed, no muscle tonus, or moribund. The behavior is scored by the same experienced observer in all experiments. The doses used are obtained from pilot dose-response experiments. The data is analyzed using appropriate statistical methods. In this method, baclofen (9.6 mg/kg), a GABA_{B} agonist, produces a marked hypothermia in GABA_{B(1)}^{+/-} and GABA_{B(1)}^{+/+} but not GABA_{B(1)}^{-/-} mice, which reaches a minimum at 60-80 min after administration, and subsequently returns towards baseline levels. The minimum temperature is about 3° C. less than the temperature of GABA_{B(1)}^{-/-} mice. Behavioral effects are also observed following the administration of baclofen to GABA_{B(1)}^{+/-} and GABA_{B(1)}^{+/+} but not GABA_{B(1)}^{/-} mice.

### Example 5

### Use of Animal Models to Assess the Efficacy of Compounds for Treating Neuropathic Pain

### Inflammatory Pain—Formalin Test

A formalin assessment test is performed according to the procedure described by Dubuisson and Dennis, Pain 1977, 4, 161-174. Fifty (50) μL of a 5% formalin solution is injected subcutaneously into the dorsal aspect of the right hind paw and the rats are then individually placed into clear observation cages. Rats are observed for a continuous period of 60 min or for periods of time corresponding to phase I (from 0 to 10 min following formalin injection) and phase II (from 30 to 50 min following formalin injection) of the formalin test. The number of flinching behaviors of the injected paw is recorded using a sampling technique in which each animal is observed for one 60-sec period during each 5-min interval. Test compound is administered 30 min or other appropriate interval prior to formalin injection.

### Inflammatory Pain—Carrageenan-Induced Acute Thermal Hyperalgesia and Edema

Paw edema and acute thermal hyperalgesia are induced by injecting 100 μL of a 1% solution of λ-carrageenan in physiological saline into the plantar surface of the right hind paw of rats. Thermal hyperalgesia is determined 2 h following carrageenan injection using a thermal paw stimulator as described by Hargreaves et al., Pain 1988, 32, 77-88. Rats are placed into plastic cubicles mounted on a glass surface maintained at 30° C. and a thermal stimulus in the form of radiant heat emitted from a focused projection bulb is then applied to the plantar surface of each hind paw. The maximum time of exposure is set to limit possible tissue damage. The elapsed time until a brisk withdrawal of the hind paw from the thermal stimulus is recorded automatically using photodiode motion sensors. The right and left hind paw of each rat is tested in three sequential trials at about 5-min intervals. Carrageenan-induced thermal hyperalgesia of paw withdrawal latency (PWLₜₕₑᵣₘₐₗ) is calculated as the mean of the two shortest latencies. Test compound is administered 30 min before assessment of thermal hyperalgesia.

The volume of paw edema is measured using water displacement with a plethysmometer 2 h following carrageenan injection by submerging the paw up to the ankle hairline (approx. 1.5 cm). The displacement of the volume is measured by a transducer and recorded. Test compound is administered at an appropriate time following carrageenan injection, such as for example, 30 min or 90 min.

### Visceral Pain

Thirty min following administration of test compound, mice receive an injection of 0.6% acetic acid in sterile water (10 mL/kg, i.p.). Mice are then placed in table-top Plexiglass observation cylinders (60 cm high×40 cm diameter) and the number of constrictions/writhes (a wave of mild constriction and elongation passing caudally along the abdominal wall, accompanied by a slight twisting of the trunk and followed by bilateral extension of the hind limbs) is recorded during the 5-20 min following acetic acid injection for a continuous observation period of 15 min.

### Neuropathic Pain—Spinal Nerve Ligation

Rats receive unilateral ligation of the lumbar 5 (L5) and lumbar 6 (L6) spinal nerves as described by Kim and Chung, Pain 1992, 50, 355-363. The left L5 and

L6 spinal nerves of the rat are isolated adjacent to the vertebral column and tightly ligated with a 5-0 silk suture distal to the dorsal root ganglia, and care is taken to avoid injury of the lumbar 4 (L4) spinal nerve. Control rats undergo the same procedure but without nerve ligation. All animals are allowed to recover for at least 1 week and not more than 3 weeks prior to assessment of mechanical allodynia. Mechanical allodynia is measure using calibrated von Frey filaments. Rats are placed into inverted plastic containers (20 cm×12.5 cm×20 cm) on top of a suspended wire mesh grid and acclimated to the test chamber for 20 min. The von Frey filaments are presented perpendicularly to the plantar surface of the selected hind paw and then held in this position for approximately 8 s with sufficient force to cause a slight bend in the filament. Positive responses include an abrupt withdrawal of the hind paw from the stimulus or flinching behavior immediately following removal of the stimulus. A 50% paw withdrawal threshold (PWT) is determined. Rats with a PWT ≦5.0 g are considered allodynic and utilized to test the analgesic activity of a test compound. The test compound is administered 30 min or other appropriate interval prior to the assessment of mechanical allodynia.

### Neuropathic Pain—Chronic Constriction Injury of the Sciatic Nerve

A model of chronic constriction injury of the sciatic nerve-induced neuropathic pain according to the method of Bennett and Xie, Pain 1988, 33, 87-107, is used. The right common sciatic nerve is isolated at mid-thigh level and loosely ligated by four chromic gut (4-0) ties separated by an interval of 1 mm. Control rats undergo the same procedure but without sciatic nerve constriction. All animals are allowed to recover for at least 2 weeks and for no more than 5 weeks prior to testing of mechanical allodynia. Allodynic PWT is assessed in the animals as described for animals with spinal nerve ligation. Only rats with a PWT ≦5.0 g are considered allodynic and utilized to evaluate the analgesic activity of a test compound. Test compound is administered 30 min or other appropriate time prior to the assessment of mechanical allodynia.

### Neuropathic Pain—Vincristine-Induced Mechanical Allodynia

A model of chemotherapy-induced neuropathic pain is produced by continuous intravenous vincristine infusion (Nozaki-Taguchi et al., Pain 2001, 93, 69-76). Anesthetized rats undergo a surgical procedure in which the jugular vein is catheterized and a vincristine-primed pump is implanted subcutaneously. Fourteen days of intravenous infusion of vincristine (30 μg/kg/day) results in systemic neuropathic pain of the animal. Control animals undergo the same surgical procedure, with physiological saline infusion. PWT of the left paw is assessed in the animals 14 days post-implantation as described for the spinal nerve ligation model. Test compound is administered 30 min or other appropriate interval prior to the test for mechanical allodynia in rats with PWT ≦5.00 g before treatment.

### Post-Operative Pain

A model of post-operative pain is performed in rats as described by Brennan et al., Pain 1996, 64, 493-501. The plantar aspect of the left hind paw is exposed through a hole in a sterile plastic drape, and a 1-cm longitudinal incision is made through the skin and fascia, starting 0.5 cm from the proximal edge of the heel and extending towards the toes. The plantaris muscle is elevated and incised longitudinally leaving the muscle origin and insertion points intact. After hemostasis by application of gently pressure, the skin is apposed with two mattress sutures using 5-0 nylon. Animals are then allowed to recover for 2 h following surgery, at which time mechanical allodynia and thermal hyperalgesia are assessed.

Effects of test compound on mechanical allodynia are assessed 30 min following administration, with PWT being examined in these animals for both the injured and non-injured paw as described for the spinal nerve ligation model with the von Frey filament systematically pointing towards the medial side of the incision. In a separate experiment, the effects of test compound on thermal hyperalgesia are assessed 30 min following administration of test compound, with PWLₜₕₑᵣₘₐₗ being determined as described for the carrageen-induced thermal hyperalgesia model with the thermal stimulus applied to the center of the incision of the paw planter aspect.

### Example 6

### Use of Animal Models to Assess the Efficacy of Prodrugs of GABA_{B} Agonists for Treating Musculoskeletal Pain

An animal model of muscle hyperalgesia described by Kehl et al., Pain 2000, 85, 333-343, can be used to assess the usefulness of a prodrug of a GABA_{B} agonist for treating musculoskeletal pain.

Male Sprague-Dawley rats are used in the study. Animals are housed for 1 week before each experiment and weigh approximately 100-150 g when carrageenan is injected. At the start of each experiment baseline forelimb and hindlimb grip force measurements are acquired. Each animal is then briefly anesthetized and carrageenan (4 mg/75 μL per triceps) or PBS vehicle (75 μL) is injected into the triceps muscles bilaterally. To determine whether grip force reduction is specifically mediated by carrageenan, various doses of carrageenan or an equal volume of PBS vehicle are injected into the triceps muscles bilaterally. The forelimb and hindlimb grip force is then measured at various intervals following the injections and compared to pre-carrageenan levels.

Measurement of forelimb grip force is made using a computerized grip force meter. The apparatus measures the neuromuscular performance of rodents as displayed in their forelimb and hindlimb grip force responses. Two separate force gauges are used to measure the responses, with one gauge for measuring forelimb grip force located at the front of the apparatus, and the other gauge, that measures hindlimb grip force, located at the rear of the apparatus. During testing, each rat is held by its tail and gently passed (about 10 cm/sec) over the wire mesh grids and the grip force measured by the strain gauges. The length of time each animal applies force to the mesh grid is determined by the animal itself, and therefore the amplitude and duration of force exerted are subject to factors, such as hyperalgesia, influencing the behavioral performance of the animal.

To test the anatomical specificity of carrageenan-evoked grip force reduction, the force measurement apparatus is modified to position both force transducers with attached wire mesh grids side-by-side at the front of the apparatus. Rats are held by their tails and gently passed (about 10 cm/sec) over the side-by-side wire mesh grids to obtain separate baseline forelimb grip force measurements from the right and left forelimbs simultaneously. Rats are then injected bilaterally with carrageenan (4 mg) or PBS (75 μL) into the triceps to obtain the following three treatment groups: (1) bilateral PBS (75 μL); (2) bilateral carrageenan (4 mg); and (3) PBS (75 μL) in one triceps and carrageenan (4 mg) in the contralateral triceps. The side selected for carrageenan injection is randomized and the observer is unaware of the treatment allocation. Bilateral grip force measurements are then obtained at intervals over the next 48 h and compared to baseline measurements.

To evaluate compounds for effectiveness in treating clinical muscle pain, baseline grip force measurements are first obtained. Carrageenan is then injected bilaterally and grip force measured 11 h later, at a time determined in the first experiment to exhibit peak reduction in grip force. Immediately after testing, an appropriate amount of a test compound is administered. Grip force is measured 30 min later and compared to the baseline levels for each animal. Test compounds that inhibit carrageenan-evoked reduction in grip force may be efficacious in treating musculoskeletal pain in humans.

### Example 7

### Intracolonic Absorption of Prodrugs of GABA_{B} Agonists in Rats

Sustained release oral dosage forms, which release drug slowly over periods of 6-24 h, generally release a significant proportion of the dose within the colon. Thus, drugs suitable for use in such dosage forms preferably exhibit good colonic absorption. The following method can be used to assess the intracolonic absorbability of GABA_{B} agonist prodrugs provided by the present disclosure in rats, and therefore the appropriateness of the GABA_{B} agonist prodrugs for use in oral sustained release dosage forms for treating neuropathic and musculoskeletal pain.

Rats were obtained commercially and were pre-cannulated in the both the ascending colon and the jugular vein. Animals were conscious at the time of the experiment. All animals were fasted overnight and until 4 hours post-dosing. (R)-Baclofen or baclofen prodrugs:
sodium 4-[(acetoxymethoxy)carbonylamino]-(3R)-(4-chlorophenyl)-butanoate;
sodium 4-[(benzoyloxymethoxy)carbonylamino]-(3R)-(4-chlorophenyl)-butanoate;
sodium 4-[(1-acetoxyisobutoxy)carbonylamino]-(3R)-(4-chlorophenyl)-butanoate;
sodium 4-[(1-isobutanoyloxyisobutoxy)carbonylamino]-(3R)-(4-chlorophenyl)-butanoate;
sodium 4-[(1-butanoyloxyisobutoxy)carbonylamino]-(3R)-(4-chlorophenyl)-butanoate;
sodium 4-[(1-butanoyloxyethoxy)carbonylamino]-(3R)-(4-chlorophenyl)-butanoate;
sodium 4-[(1-isobutanoyloxyethoxy)carbonylamino]-(3R)-(4-chlorophenyl)-butanoate;
sodium 4-[(1-benzoyloxyethoxy)carbonylamino]-(3R)-(4-chlorophenyl)-butanoate;
sodium 4-[(2,2-diethoxypropanoyloxymethoxy)carbonylamino]-(3R)-(4-chlorophenyl)-butanoate;
sodium 4-{[(1S)-isobutanoyloxyisobutoxy]carbonylamino}-(3R)-(4-chlorophenyl)-butanoate;
sodium 4-{[(1R)-isobutanoyloxyisobutoxy]carbonylamino}-(3R)-(4-chlorophenyl)-butanoate; and
4-{[(1S)-isobutanoyloxyisobutoxy]carbonylamino}-(3R)-(4-chlorophenyl)-butanoic acid;
   were independently administered as a solution (in water or PEG 400) directly into the colon via the cannula at a dose equivalent to 10 mg of baclofen equivalents per kg body weight. Blood samples (0.5 mL) were obtained from the jugular cannula at intervals over 8 hours and quenched immediately by addition of methanol to prevent further conversion of the prodrug. Blood samples were analyzed as described according to the following procedure.

Rat blood was collected at different time points and 100 μL of blood was added into an Eppendorf tube containing 300 μL of methanol and vortexed to mix immediately. Twenty (20) μL of p-chlorophenylalanine was added as an internal standard. Three-hundred μL of methanol was added into each tube followed by 20 μL of p-chlorophenylalanine. Ninety (90) μL of blank rat blood was added to each tube and mix. Then 10 μL of a baclofen standard solution (0.04, 0.2, 1, 5, 25, 100 μg/mL) was added to make up a final calibration standard (0.004, 0.02, 0.1, 0.5, 2.5, and 10 μg/mL). Samples were vortexed and centrifuged at 14,000 rpm for 10 min. Supernatant was taken for LC/MS/MS analysis.

An API 2000 LC/MS/MS spectrometer equipped with Shimadzu 10ADVp binary pumps and a CTC HTS-PAL autosampler was used in the analysis. A Phenomenex hydro-RP 4.6×50 mm column was used during the analysis. The mobile phase was water with 0.1% formic acid (A) and acetonitrile with 0.1% formic acid (B). The gradient condition was: 10% B for 0.5 min, then to 95% B in 2.5 min, then maintained at 95% B for 1.5 min. The mobile phase was returned to 10% B for 2 min. A TurboIonSpray source was used on the API 2000. The analysis was done in positive ion mode and using optimal MRM transitions for each compound. Ten (10) μL of the samples were injected. The peaks were integrated using Analyst 1.2 quantitation software.

Following colonic administration of prodrugs:
sodium 4-[(benzoyloxymethoxy)carbonylamino]-(3R)-(4-chlorophenyl)-butanoate;
sodium 4-[(1-acetoxyisobutoxy)carbonylamino]-(3R)-(4-chlorophenyl)-butanoate;
sodium 4-[(1-isobutanoyloxyisobutoxy)carbonylamino]-(3R)-(4-chlorophenyl)-butanoate;
sodium 4-[(1-butanoyloxyisobutoxy)carbonylamino]-(3R)-(4-chlorophenyl)-butanoate;
sodium 4-[(1-butanoyloxyethoxy)carbonylamino]-(3R)-(4-chlorophenyl)-butanoate;
sodium 4-[(1-isobutanoyloxyethoxy)carbonylamino]-(3R)-(4-chlorophenyl)-butanoate;
sodium 4-[(1-benzoyloxyethoxy)carbonylamino]-(3R)-(4-chlorophenyl)-butanoate;
sodium 4-[(2,2-diethoxypropanoyloxymethoxy)carbonylamino]-(3R)-(4-chlorophenyl)-butanoate
sodium 4-{[(1S)-isobutanoyloxyisobutoxy]carbonylamino}-(3R)-(4-chlorophenyl)-butanoate;
sodium 4-{[(1R)-isobutanoyloxyisobutoxy]carbonylamino}-(3R)-(4-chlorophenyl)-butanoate; and
4-{[(1S)-isobutanoyloxyisobutoxy]carbonylamino}-(3R)-(4-chlorophenyl)-butanoic acid;

the maximum plasma concentrations of (R)-baclofen (Cₘₐₓ), as well as the area under the baclofen plasma concentration vs. time curves (AUC) were significantly greater (>2-fold) than that produced following colonic administration of (R)-baclofen itself. This data demonstrates that these compounds may be formulated as compositions suitable for enhanced intracolonic absorption and/or effective oral sustained release of GABA_{B} receptor agonists for treating neuropathic or musculoskeletal pain.

### Example 8

### Intracolonic Absorption of Prodrugs of GABA_{B} Agonists in Cynomolgus Monkeys

The following method can be used to assess the intracolonic absorbability of GABA_{B} agonist prodrugs provided by the present disclosure in Cymologous monkeys, and therefore the appropriateness of the GABA_{B} agonist prodrugs for use in oral sustained release dosage forms for treating neuropathic and musculoskeletal pain.

(R)-Baclofen hydrochloride salt and (R)-baclofen prodrugs (5 mg (R)-baclofen-eq/kg) were administered to groups of four male cynomolgus monkeys as either aqueous solutions or suspensions in 0.5% methyl cellulose/0.1% Tween-80 via bolus injection directly into the colon via an indwelling cannula. For colonic delivery a flexible French catheter was inserted into the rectum of each monkey and extended to the proximal colon (approx. 16 inches) using fluoroscopy. Monkeys were lightly sedated by administration of Telazol/ketamine during dosing. A washout period of at least 5 to 7 days was allowed between treatments. Following dosing, blood samples were obtained at intervals over 24 hours and were immediately quenched and processed for plasma at 4° C. All plasma samples were subsequently analyzed for (R)-baclofen and intact prodrug using the LC/MS/MS assay described above. Following colonic administration of prodrugs sodium 4-[(benzoyloxymethoxy)carbonylamino]-(3R)-(4-chlorophenyl)-butanoate; benzyl 4-[(1-acetoxyisobutoxy)carbonylamino]-(3R)-(4-chlorophenyl)-butanoate; sodium 4-[(1-benzoyloxyethoxy)carbonylamino]-(3R)-(4-chlorophenyl)-butanoate; 4-[(2,2-diethoxypropanoyloxymethoxy)carbonylamino]-(3R)-(4-chlorophenyl)-butanoate; the maximum plasma concentrations of (R)-baclofen (Cₘₐₓ), as well as the area under the baclofen plasma concentration vs. time curves (AUC) were significantly greater (>2-fold) than that produced from colonic administration of (R)-baclofen itself, while colonic administration of sodium 4-{[(1S)-isobutanoyloxyisobutoxy]carbonylamino}-(3R)-(4-chlorophenyl)-butanoate; sodium 4-{[(1R)-isobutanoyloxyisobutoxy]carbonylamino)-(3R)-(4-chlorophenyl)-butanoate; 4-{[(1S)-isobutanoyloxyisobutoxy]carbonylamino}-(3R)-(4-chlorophenyl)-butanoic acid; produced (R)-baclofen exposures that were greater than 10-fold that produced from colonic administration of (R)-baclofen itself. This data demonstrates that these compounds may be formulated as compositions suitable for enhanced intracolonic absorption and/or effective oral sustained release of GABA_{B} agonists for treating neuropathic and musculoskeletal pain.

### Example 9

### Oral Bioavailability of Intracolonically Absorbable GABA_{B} Agonists in Cymologous Monkeys

The following method can be used to determine the oral bioavailability of GABA_{B} agonist prodrugs provided by the present disclosure in Cymologous monkeys, and therefore the appropriateness of the GABA_{B} agonist prodrugs for use in oral sustained release dosage forms for treating neuropathic and musculoskeletal pain.

The (R)-baclofen prodrugs sodium 4-{[(1S)-isobutanoyloxyisobutoxy]carbonylamino}-(3R)-(4-chlorophenyl)-butanoate and 4-{[(1S)-isobutanoyloxyisobutoxy]carbonylamino}-(3R)-(4-chlorophenyl)-butanoic acid (5 mg (R)-baclofen-eq/kg) were administered by oral gavage to groups of four male cynomolgus monkeys as either an aqueous solution or suspension in 0.5% methylcellulose/0.1% Tween-80 respectively. Following dosing, blood samples were obtained at intervals over 24 hours and were immediately quenched and processed for plasma at 4° C. All plasma samples were subsequently analyzed for (R)-baclofen and intact prodrug using the LC/MS/MS assay described above. The oral bioavailability of both prodrugs sodium 4-{[(1S)-isobutanoyloxyisobutoxy]carbonylamino}-(3R)-(4-chlorophenyl)-butanoate and 4-{[(1S)-isobutanoyloxyisobutoxy]carbonylamino}-(3R)-(4-chlorophenyl)-butanoic acid as (R)-baclofen was determined to be greater than 80%.

### Example 10

### Pharmacokinetics of (R)-Baclofen in Human Patients Following Oral Administration of Sustained Release Oral Dosage Forms Comprising Compound (4)

The pharmacokinetics of (R)-baclofen in healthy human patients following oral administration of controlled release (CR) capsules comprising compound (4) was determined. The preparation of controlled release capsules is described in Leung et al., US 2008/0206332, which is incorporated by reference herein in its entirety. A CR capsule comprised particles of 20/25 sugar spheres coated with compound (4) and Plasidone® K29/32 Povidone, and having an overcoat of Eudragit® RL 100.

Fasted human patients were randomized to receive single oral doses of CR capsules or matching placebo in a double-blind fashion. The study investigated 6 dose levels of compound (4), 10, 20, 30, 40, 60, and 80 mg, in capsules comprising controlled release particles and comprising 10 mg compound (4). Six (6) groups of 10 subjects each were enrolled sequentially (10 subjects per dose level). Eight subjects in each dose group received CR capsules and two received placebo.

Blood samples were collected from patients prior to dosing and at 0.5, 1, 1.5, 2, 3, 4, 5, 6, 8, 10, 12, 14, 18, 24, 30, and 36 hours post-dosing for all treatments. Blood sample aliquots were quenched immediately with methanol to prevent further hydrolysis of compound (4). Blood sample aliquots were stored in a freezer at −70° C. The blood sample aliquots were analyzed for (R)-baclofen and compound (4) in whole blood supernatant using sensitive and specific LC-MS/MS methods.

Concentration data for (R)-baclofen and compound (4) in blood were analyzed by noncompartmental methods using WinNonlin™ Software version 4.1 (Pharsight Corporation, Mountain View, Calif.). Concentration data and pharmacokinetics parameters were plotted using SigmaPlot™ version 9.0 (Systat Software Inc., Point Richmond, Calif.). Actual time points were used for the calculation of pharmacokinetic parameters. The maximum concentration (Cₘₐₓ) and time to Cₘₐₓ(Tₘₐₓ) were obtained by observation. The apparent elimination half-life (T_{1/2}) was determined by linear regression of three or more log-transformed data points in the terminal phase. The area under the concentration versus time curve (AUC) was determined by the linear trapezoidal method using concentration data over the dosing interval. The AUC value extrapolated to infinity (AUC_{inf}) was calculated as:

AUC_{inf}=AUC₍₀₋ₜₗₐₛₜ₎+Cₗₐₛₜ/λ_{z}

where tₗₐₛₜ is the time of the last quantifiable concentration (Cₗₐₛₜ) and λ_{z} is the rate constant of the apparent terminal elimination phase. Using the data from doses 10, 20, 30, 40, 60, and 80 mg, linear regression models were fit for AUC_{inf} versus dose and for Cₘₐₓ versus dose using SAS™ version 9.1 for Windows (SAS Institute, Cary, N.C.). In both models, the dose effect was parameterized using orthogonal polynomial coefficients for unequally spaced values.

A summary of the pharmacokinetic parameters for (R)-baclofen for different doses of (R)-baclofen prodrug (4) is provided in Table 1.

TABLE 1
Pharmacokinetic Parameters of (R)-Baclofen Following
Oral Administration of CR Capsules to Human Patients.

| Dose (mg) | 10 | | 20 | | 30 | | 40 | | 60 | | 80 | |
|---|---|---|---|---|---|---|---|---|---|---|---|---|
| Cₘₐₓ | 23 | (10) | 35 | (17) | 63 | (19) | 82 | (49) | 139 | (56) | 193 | (89) |
| (ng/mL) | | | | | | | | | | | | |
| Cₘₐₓ/dose | 2.3 | | 1.7 | | 2.1 | | 2.1 | | 2.3 | | 2.4 | |
| Cₘₐₓ/C₁₂ | 2.6 | | 2.7 | | 2.3 | | 2.2 | | 2.3 | | 2.8 | |
| Tₘₐₓ (h) | 5.0 | (3.8) | 4.1 | (1.1) | 4.8 | (0.9) | 4.5 | (1.2) | 3.9 | (1.1) | 4.0 | (1.1) |
| T_{1/2} (h) | 10.3 | (3.6) | 9.6 | (1.7) | 9.3 | (2.7) | 11.3 | (4.7) | 10.5 | (2.6) | 9.7 | (1.0) |
| AUC_{inf} | 243 | (66) | 338 | (83) | 810 | (169) | 1020 | (300) | 1540 | (603) | 2020 | (787) |
| (ng · h/mL) | | | | | | | | | | | | |
| AUC_{inf}/dose | 24 | | 17 | | 27 | | 26 | | 26 | | 26 | |
| F (%) | 31 | (7) | 33 | (10) | 33 | (7) | 28 | (9) | 35 | (6) | 34 | (18) |

The pharmacokinetics of (R)-baclofen in healthy human patients following oral administration of tablet dosage forms comprising 20 mg of compound (4) was also determined. The preparation of tablet dosage forms is described in Leung et al., US 2008/0206332, which is incorporated by reference herein in its entirety.

Fed or fasted human patients were randomized to receive single oral doses of 20 mg compound (4) as oral tablet dosage forms comprising 10 mg compound (4). Samples were obtained and analyzed as previously described. Matrix tablet dosage forms comprised 10.00 mg compound (4), 42.75 mg Eudragit® RL 30D, 107.25 mg microcrystalline cellulose, 88.75 mg hydroxypropylmethyl cellulose, and 1.25 mg magnesium stearate.

The blood concentration and pharmacokinetic parameters for (R)-baclofen following oral administration of tablet dosage forms comprising compound (4) to healthy human patients is summarized in Table 2 and Table 3. In the tables, Cₘₐₓ is the maximum concentration, C₁₂ is the concentration at 12 hours post dose, Tₘₐₓ is the time to Cₘₐₓ, AUC is the area under the drug concentration-time curve calculated using linear trapezoidal summation form time zero to time tlast, where tlast is the time of the last measurable concentration (Cₜ).

TABLE 2
Pharmacokinetic Parameters for (R)-Baclofen in Blood After Oral
Dosing of a Sustained Release Tablet Formulation Comprising 20
mg (R)-Baclofen Prodrug (4) in Fasted Healthy Subjects.

| | Tₘₐₓ | Cₘₐₓ | AUC₍₀₋ₗₐₛₜ₎ | C_{12 hr} | |
|---|---|---|---|---|---|
| | (h) | (ng/mL) | (ng · h/mL) | (ng/mL) | Cₘₐₓ/C₁₂ |
| Mean | 4.10 | 33.4 | 499 | 17.2 | 2.14 |
| SD | 2.33 | 12.3 | 134 | 8.99 | 0.82 |

TABLE 3
Pharmacokinetic Parameters for (R)-Baclofen in Blood After Oral
Dosing of a Sustained Release Tablet Formulation Comprising
20 mg (R)-Baclofen Prodrug (4) in Fed Healthy Subjects.

| | Tₘₐₓ | Cₘₐₓ | AUC₍₀₋ₗₐₛₜ₎ | C_{12 hr} | |
|---|---|---|---|---|---|
| | (h) | (ng/mL) | (ng · h/mL) | (ng/mL) | Cₘₐₓ/C₁₂ |
| Mean | 8.00 | 66.0 | 830 | 48.8 | 1.61 |
| SD | 2.67 | 18.5 | 281 | 26.7 | 0.71 |

### Example 11

### Clinical Trial to Determine Safety, Tolerability, and Efficacy of GABA_{B} Agonists in Treating Acute Neck and Lower Back Spasms

A multi-center, randomized, double-blind, placebo-controlled, parallel group study of the safety, tolerability, and efficacy of a prodrug of a GABA_{B} agonist provided by the present disclosure, such as for example, compound (4) in patients with acute back muscle spasm in the lumbar, thoracic and/or cervical regions. Primary outcome measures can include: the incidence of treatment-emergent adverse events; changes from baseline in vital signs, electrocardiograms, and safety laboratory tests; and efficacy assessment.

Each of three dose levels are evaluated in a sequential design. In period 1 approximately 60 adult male and female patients are enrolled who will be randomized into one of three treatment arms (1:1:1; 20 mg, 30 mg or placebo BID). Following enrollment of Period 1, approximately 60 patients are randomized into one of four treatment arms (1:1:3:1; 20 mg, 30 mg, 40 mg, or placebo BID) for a total of approximately 120 patients in the study. Enrollment in Period 2 proceeds upon the study completion of the last patient enrolled in Period 1, and only if it is determined that safety and tolerability in Period 1 are acceptable. This design is selected in order to maintain blinding to treatment while deferring exposure to the 40 mg BID dose until the safety and tolerability of lower doses has been assessed. R-baclofen exposures after dosing with 40 mg BID (of compound (4)) in the study are predicted to be similar to those after dosing with racemic baclofen 20 mg QID, which was found to be effective in treatment of acute muscle spasms in the back (Dapas et al., Spine 1985, 10(4), 345-349). Doses lower than 40 mg BID are also studied. The total study duration will be approximately 14 days not including screening.

The following outcome measure to assess treatment efficacy may be determined:

Proportion of subjects with a rating of very good and excellent in subject's rating of medication helpfulness. During their clinic visits at Day 4, Day 10, and Day 14, subjects will assess the helpfulness of their study drug in improving their condition using a 5-point categorical rating scale: 0=poor, 1=fair, 2=good, 3=very good, 4=excellent.

Proportion of subjects with moderate to marked improvement in subject-rated clinical global impression of change (CGI-C). During their clinic visits at Day 4, Day 10, and Day 14, subjects will assess their clinical global impression of change from baseline using a 5-point categorical rating scale: 0=worsening, 1=no change, 2=mild improvement; 3=moderate improvement, and 4=marked improvement.

Change in subject-rated relief of starting backache, using an electronic subject study diary. Because the twice daily dosing regimen for os a sustained release oral dosage form comprising a prodrug of a GABA_{B} agonist such as compound (4) is expected to result in sustained efficacy through the night, assessment of pain using the subject-rated relief of starting backache will occur twice daily: prior to the first dose of study medication on each dosing day (with regard to symptoms occurring during the previous night), and 3 hours after the second dose of study medication (with regard to symptoms occurring during the previous day).

Change in subject-rated daytime drowsiness. Using an electronic subject study diary, subjects will rate their overall daytime drowsiness once per evening using a 5-point categorical scale: 1=No Drowsiness, 2=Very Little Drowsiness, 3=Some Drowsiness, 4=A Lot of Drowsiness, 5=Extreme Drowsiness.

Subject-rated restriction of movement. During the subject's clinic visits at baseline, Day 4, Day 10, and Day 14, subject will evaluate and rate the subject's range of motion based on a 5-point categorical rating scale: 0=worsening, 1=no change, 2=mild improvement; 3=moderate improvement, and 4=marked improvement.

Change in physician-rated severity of muscle spasms. During the subject's clinic visits at baseline, Day 4, Day 10, and Day 14, physician will evaluate and rate the subject's severity of muscle spasm based on a 5-point rating scale: 0=no hardness, 1=mild, 2=moderate with borders of increased consistency, 3=moderately severe with sharply defined borders, 4=severe−boardlike hardness of muscles.

Change in subject-rated Pain Disability Index. During their clinic visits at baseline, Day 4, Day 10, and Day 14, subjects will assess their overall disability using a 10-point rating system of 7 functional activities.

Change in Roland Morris Disability Questionnaire (RMDQ). During their clinic visits at baseline, Day 4, Day 10, and Day 14, subjects will complete the RMDQ.

Other protocols for assessing treatment of acute lower bask spasms are known (Ralph et al., Current Medical Research and Opinions 2008, 24(2), 551-558).

Finally, it should be noted that there are alternative ways of implementing the disclosures contained herein. Accordingly, the present embodiments are to be considered as illustrative and not restrictive, and the claims are not to be limited to the details given herein, but may be modified within the scope and equivalents thereof.

What is claimed is:

1. A method of treating neuropathic pain in a patient comprising orally administering to a patient in need of such treatment a therapeutically effective dose of a compound of Formula (I): 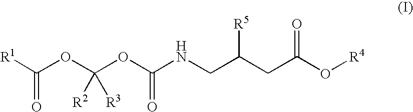
or a pharmaceutically acceptable salt thereof, wherein:
R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, cycloheteroalkyl, and substituted cycloheteroalkyl ring;
R⁴ is chosen from hydrogen, alkyl, substituted alkyl, aryl, substituted aryl, arylalkyl, substituted arylalkyl, aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, and trialkylsilyl; and
R⁵ is chosen from substituted aryl, heteroaryl, and substituted heteroaryl.

2. The method of claim 1, wherein the compound is a compound of Formula (III): 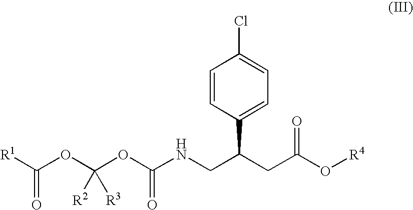
or a pharmaceutically acceptable salt thereof, wherein:
R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, cycloheteroalkyl, and substituted cycloheteroalkyl ring; and
R⁴ is chosen from hydrogen, alkyl, substituted alkyl, aryl, substituted aryl, arylalkyl, substituted arylalkyl, aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, and trialkylsilyl.

3. The method of claim 2, wherein the compound is (3R)-4-{[(1S)-2-methyl-1-(2-methylpropanoyloxy)propoxy]carbonylamino}-3-(4-chlorophenyl)butanoic acid or a pharmaceutically acceptable salt thereof.

4. The method of claim 2, wherein the therapeutically effective dose comprises about 1 mg-equivalent of (R)-baclofen to about 100 mg-equivalent of (R)-baclofen.

5. The method of claim 2, wherein the therapeutically effective dose comprises about 20 mg-equivalents of (R)-baclofen per day to about 100 mg-equivalents of (R)-baclofen per day.

6. The method of claim 1, wherein the therapeutically effective dose is less than a dose that causes moderate sedation and impairment of motor activity in the patient.

7. The method of claim 1, wherein the neuropathic pain is chosen from post-herpetic neuralgia, peripheral neuropathy, trigeminal neuralgia, painful diabetic neuropathy, HIV-related neuropathic pain, cancer-related pain, and fibromyalgia.

8. The method of claim 1, wherein orally administering comprises administering a sustained release oral dosage form.

9. The method of claim 1, wherein the method further comprise administering a second compound useful for treating neuropathic pain.

10. A method of treating musculoskeletal pain in a patient comprising orally administering to a patient in need of such treatment a therapeutically effective dose of a compound of Formula (I): 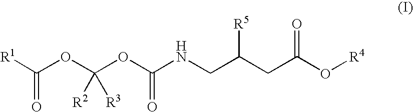
or a pharmaceutically acceptable salt thereof, wherein:
R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, cycloheteroalkyl, and substituted cycloheteroalkyl ring;
R⁴ is chosen from hydrogen, alkyl, substituted alkyl, aryl, substituted aryl, arylalkyl, substituted arylalkyl, aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, and trialkylsilyl; and
R⁵ is chosen from substituted aryl, heteroaryl, and substituted heteroaryl.

11. The method of claim 10, wherein the compound is a compound of Formula (III): 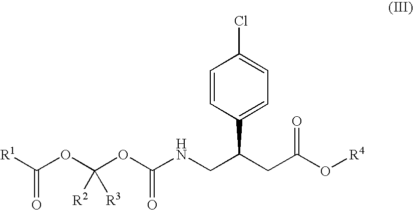
or a pharmaceutically acceptable salt thereof; wherein:
R¹ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, cycloheteroalkyl, and substituted cycloheteroalkyl ring; and
R⁴ is chosen from hydrogen, alkyl, substituted alkyl, aryl, substituted aryl, arylalkyl, substituted arylalkyl, aryldialkylsilyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, and trialkylsilyl.

12. The method of claim 11, wherein the compound is (3R)-4-{[(1S)-2-methyl-1-(2-methylpropanoyloxy)propoxy]carbonylamino}-3-(4-chlorophenyl)butanoic acid or a pharmaceutically acceptable salt thereof.

13. The method of claim 11, wherein the therapeutically effective dose comprises about 1 mg-equivalent of (R)-baclofen to about 100 mg-equivalent of (R)-baclofen.

14. The method of claim 11, wherein the therapeutically effective dose comprises about 20 mg-equivalents of (R)-baclofen per day to about 100 mg-equivalents of (R)-baclofen per day.

15. The method of claim 10, wherein the therapeutically effective dose is less than a dose that causes moderate sedation and impairment of motor activity in the patient.

16. The method of claim 10, wherein the musculoskeletal pain is tension headache.

17. The method of claim 10, wherein orally administering comprises administering a sustained release oral dosage form.

18. The method of claim 10, wherein the method further comprises administering a second compound useful for treating musculoskeletal pain.

19. The method of claim 10, wherein the musculoskeletal pain is back pain.

20. The method of claim 19, wherein the back pain is acute low back pain.

21. The method of claim 10, wherein the musculoskeletal pain is associated with muscle spasm.

22. The method of claim 21, wherein the associated muscle spasm is acute back muscle spasm.

23. The method of claim 22, wherein the acute back muscle spasm is acute lower back muscle spasm.

24. The method of any one of claims 2 and 11, which provides a maximum plasma concentration (Cₘₐₓ) of less than 200 ng/mL of (R)-baclofen and a total plasma (R)-baclofen exposure of at least 1,500 ng-hr/mL (AUC₀₋₂₄).

25. The method of any one of claims 2 and 11, which provides a maximum plasma concentration (Cₘₐₓ) of less than 150 ng/mL of (R)-baclofen and a total plasma (R)-baclofen exposure of at least 1,000 ng-hr/mL (AUC₀₋₂₄).
